(19) United States
(12) Patent Application Publication
(10) Pub. No.: US 2020/0342981 A1
(43) Pub. Date: Oct. 29, 2020

(54) REVERSE FLOW APPOINTMENT SCHEDULING

(71) Applicant: OpenMed, Inc., Miami, FL (US)

(72) Inventor: Aaron KAUFMAN, Miami, FL (US)

(21) Appl. No.: 16/855,657

(22) Filed: Apr. 22, 2020

Related U.S. Application Data
(60) Provisional application No. 62/837,308, filed on Apr. 23, 2019.

(51) Int. Cl. G16H 40/20; G06Q 10/10

(52) U.S. Cl. CPC G16H 40/20; G06Q 10/1095

(57) ABSTRACT

A reverse flow appointment scheduling arrangement, implemented on a computer, which includes gathering information regarding a consumer, analyzing the user information to determine if a predetermined time has passed since the consumer has had an appointment with a provider for a predetermined service, preparing a list of available appointment times which are available for the provider to meet with the consumer to provide the predetermined service, preparing a notification to the consumer with the list of available appointment times, and sending the notification to the consumer to request that the consumer schedules an appointment by selecting one of the appointment times from the list.

### CROSS REFERENCE TO RELATED APPLICATIONS

The present application incorporates in its entirety by reference U.S. application Ser. No. 14/196,193 filed Mar. 4, 2014, entitled “Appointment Schedule”.

### FIELD OF THE INVENTION

The present invention generally relates to appointment scheduling, and more particularly, to methods, systems and/or services that schedule appointments as initiated by the provider of the services.

### BACKGROUND

Healthcare costs are out of control. Since 1970, for example, U.S. healthcare costs have risen an average of 17.5 percent every year resulting in 17 percent of GDP spent on healthcare. That gap has been growing for more than 50 years, and by 2026, the proportion will likely be 20 percent, or $5.7 trillion. The average American spends more than $10,000 per year on healthcare, about twice the amount that citizens of other wealthy countries pay. Within the next six years, the system will not be able to sustain itself if we do not reverse these trends.

To combat these rising costs, health plans and the Center for Medicare and Medicaid Services or CMS, are exchanging their payment model to from fee for service to value-based care. In value-based care, healthcare providers' payments depend on the healthy outcomes of their patients, as opposed to getting paid for specific services. And like any type of insurance, increasing the risk pool decreases each subscriber's cost. So to achieve those savings, providers and healthcare plans, accountable care organizations, and CMS have teamed up and agreed to share their risks with plans where providers are rewarded for reducing medical spending while increasing their quality of care.

One type of value-based care that has been around for several years is Medicare Part C, also known as Medicare Advantage. There are over 21.3 million patients covered under the Advantage program. In it, wellness becomes the key to health outcomes for patients and to financial rewards to health plans, managed service organizations, or MSOs, and providers. In Medicare Advantage, the largest value-based care program in the US, financial rewards depend on 1) Twice-yearly risk assessments that determine a patient's risk-adjusted factor score, which in turn sets the fee paid by Medicare, and 2) completing required treatments based on the assessments. Plans and providers benefit in many ways in this two-component model, as CMS has determined that they are the most effective way to bend the quality of care and cost curves toward sustainability.

Now, imagine a patient with a risk score that is too low, and they wind up having major medical expenses. The service fund could be devastated. And even when risk scores are accurate, if patients do not adhere to their care plans and their risk scores become less accurate over time, their hospitalization expenses could also drain the service fund. Accordingly, value-based care lowers expenses through better patient management and determining accurate risk levels.

CMS has shown through big-data studies and other research that healthcare provided by PCP in-person appointments reduces major healthcare spending trends and increases the wellness of patients, as long as the care involves key measurements, diagnostics, and appropriate preventative procedures. The in-person appointments are critical for assessments and treatment plans for every patient with or without a known illness. If done in a timely way, it is possible to achieve high-quality care at low cost.

In scheduling appointments, an individual or an organization may use a variety of different devices (e.g., telephone, computer, etc.) and methods to request and schedule appointments. For example, a patient may schedule an appointment using a telephone to request an appointment with a provider of services, e.g., doctor. Based on the provider's availability, e.g., schedule, the requested appointment may be accepted or, alternatively, when the appointment time is unavailable, a different time can be suggested and accepted. This can be a cumbersome and costly process, particularly for the provider. For example, the provider may need a dedicated person for scheduling appointments thereby increasing overhead costs significantly.

Also, if a requested time is not available, then the patient may have to contact another provider, repeating this same process. As such, scheduling an appointment (e.g., a doctor's appointment) may be time consuming, requiring contacting more than one provider before an appointment request is acceptable to all involved parties. This may thus include the cumbersome process of finding other providers within a certain geographical area, or a provider that has an acceptable reputation or expertise, etc. To find such a provider, the consumer may have to consult with insurance companies, friends, colleagues or a host of other avenues, all of which are time consuming and even at times frustrating.

Additionally, once an appointment is scheduled, changing the appointment time may be difficult. This includes, for example, contacting the provider and requesting another appointment time which may or may not be available. Also, in many known arrangements, if the appointment is not canceled within a certain time period, a penalty may be charged to the consumer. This is typically termed a cancelation fee. So, it is imperative for the consumer to reschedule an appointment within a certain time frame, which is not always possible or even practical. For example, an emergency can arise within the certain time period. In this scenario, there may be no way to avoid the cancelation fee.

Accordingly, providers spend too much time trying to see massive numbers of patients in their offices to make up for the dwindling reimbursements from fee-for-service operations. Too much time, because they typically manage by placing phone calls and recording results in spread-sheets and/or dedicated reporting systems. Practically none of them have the tools to switch their business from fee-for-service to value-based care. Health plans and CMS are demanding too much from providers without providing them the scale and compensation they need.

### SUMMARY

In recent years, healthcare plans have spent billions on analytical and reporting platforms to identify at-risk patients. The trickier part, however, seems to be getting patients in to see a provider and managing their adherence to the plans' requirements. For example, a 67-year-old male with diabetes and congestive heart failure on a Medicare Advantage Plan requires regular BMI measurements and HbA1c diagnostics. This patient must also have two risk assessments a year, as does every one of the 21.3 million Medicare Advantage Plan members. This is known as a six-month care gap. These risk assessments are key to value-based care because they uncover prior and current conditions. Ultimately it sets the level of compensation from CMS & Health Plans to providers, by confirming a members' wellness.

So, what happens if the patient skips one of his required in-person visits? The patient's health plan repeatedly reminds him and his provider of his appointments. A representative of the patient's health plan also meets with his provider to find out the best ways to get him and other patients they have with this provider to schedule and keep their appointments. The costs and time that health plans bear to ensure that their patients keep their value-based care appointments are too high. There are millions of patients like this in each health plan, so even a small percentage of them can greatly affect the costs borne by all.

To this end, the present invention scales value-based care work. Using the processes and platforms described herein, a connected provider scheduling platform is provided such that health plans can import raw analytical data and schedule appointments and remind patients of them within the platform described herein, i.e., Reverse Scheduling™. More specifically, by implementing the Reverse Scheduling platform a health-plan or provider can select a part of their member population through various filters and views, and then schedule their appointments appropriately (for example: a filter could be Medicare Part C patients with diabetes and a six-month lapsed care gap for a HbA1c test).

Also, by implementing the Reverse Scheduling platform, once a health plan identifies its at-risk patients in need of an appointment, it can use the processes and platform described herein to send appointment-scheduling messages to them through different channels: SMS, email, in-app, and interactive voice response. The patient can respond via the same channel to confirm the appointment. If the suggested times do not work for the patient, they can always request different times through the processes and platform described herein (also referred hereinafter as the “app” or Reverse Scheduling platform).

Reverse scheduling as described herein simplifies the management of health plan service funds in many ways. Scheduling patients' twice-a-year risk assessments and reducing patients' adherence issues can all be done by using the Reverse Scheduling platform. The Reverse Scheduling platform processes appointments automatically and successfully onboards providers to respond to the appointments, and most importantly, book patients' appointments. The Reverse Scheduling platform can handle millions of patient bookings every day.

To change the fundamental behavior of the current system, the Reverse Scheduling platform starts the provider with an automatic and scalable platform rather than manual activity. Real appointments between patients and providers, discussing and treating their health, capturing measures, and changing behaviors is the only way to achieve the fundamental shift toward more positive outcomes and significantly reduced healthcare costs. The Reverse Scheduling platform builds a comprehensive data structure that can include every provider in the U.S., or even around the world. The Reverse Scheduling platform also can encompass all the various types of health insurance plans every provider accepts and captures providers from patient demand into a system that does not require integration using the processes described herein.

The Reverse Scheduling platform is able to connect any patient with a provider, whether the provider knows about the Reverse Scheduling platform or not, during the initial contact. Going back to the above example, when a patient requests an appointment from his provider for a particular date and time, the Reverse Scheduling platform captures the provider by this very appointment request. If the provider does not respond to the automated requests from the Reverse Scheduling platform, the Reverse Scheduling platform can contact the provider through an onboarding call center, which is used primarily only for the first contact with a provider. The request guides the provider onto the Reverse Scheduling platform. If the request does not fit in the provider's schedule, they can simply respond with times that do fit. Once the provider is on the Reverse Scheduling platform, they are educated about services, like direct EMR integration, payment collection, campaigns, and much more. The Reverse Scheduling platform includes APIs that can be leveraged by any party. Any party can add appointment scheduling to their Application, especially, health plan Apps, MSOs, or even HR vendors, employer benefits systems, for instance.

In embodiments, the Reverse Scheduling platform is a reverse flow appointment scheduling system and processes to facilitate reverse flow appointment scheduling utilizing the infrastructure taught in U.S. application Ser. No. 14/196,193. In embodiments, medical or other types of appointments or services are started and/or requested and/or scheduled from a third party, i.e., not the consumer of the services (e.g., not initiated from the patient). The third party can be, amongst other parties, an insurance company, HMO, a doctor or doctor's office, a network provider, a healthcare plan, a primary or secondary care provider, pharmacist or other medical adherence party, or a contractually obligated party to the patient and/or service provider, etc.

As noted herein, a practical advantage of the reverse flow appointment scheduling system and processes is that it encourages patients (or, in other service industries, consumers in general) to go to their healthcare service provider in a timely manner, which, research shows, greatly reduces illness and overall healthcare costs to the consumer and/or government entities paying for care) by catching warning signs early and/or providing proactive medical care. This not only greatly benefits patients, but also assists both healthcare providers and third parties, such as insurance companies, hospitals, HMOs, etc., in managing and determining “care gaps” in regard to their covered patients. In other words, it allows both service providers and/or other third parties to determine when it is desirable, or even essential, for a patient to see an appropriate healthcare provider (care giver).

In conjunction with the flowcharts provided in the attached drawings, one or more interfaces, similar to those disclosed in U.S. application Ser. No. 14/196,193 can be used for the provider/third-party (hereinafter which is to be understood as not the consumer (patient) of the services) to navigate on a reverse scheduling application for carrying out the reverse scheduling flowcharts shown in the attached drawings. For example, in the healthcare field, OpenMed™ Web Reverse Scheduling platform (e.g., Reverse Scheduling platform or its processes and/or systems incorporated into a third party app) can be utilized to implement the reverse scheduling flowchart shown in the attached drawings and described in more detail herein.

With regard to the interfaces, as shown in FIGS. 6-26, a first interface can be used to build a filter on a population based on a condition which the provider/third party wishes to schedule for. Other interfaces could be used to show and schedule available times for appointments. And, as can be appreciated from the attached flowcharts and other figures described herein, the illustrated reverse scheduling process provides for tracking the response of patients, after sending notifications to them. The reverse scheduling process also allows for tracking activity regarding which providers are being used, which specialties are frequently utilized, etc. This tracking can be done, for example, based on ZIP Codes or other information filtered by the third party, etc.

Moreover, the attached flowcharts can be provided as event driven events, so that real-time changes with regard to notifying and/or scheduling patients (for example, if a new patient registers). The event driven features can cause modifications of the patient information database so that further appropriate notices can be sent in real time, or appointments can be suggested and/or scheduled, initiated from the third party. Another example would be allowing for real time notifications if a new drug becomes available for a certain group of patients, etc. Accordingly, by using the reverse scheduling features of the reverse flow appointment scheduling system and processes, a selected or appropriate group of patients can be quickly notified of the arrival of a new drug (or a shipment of a new supply of an regularly prescribed drug) or the need to see a care provider for certain medical conditions, and be advised to schedule an appointment to the service provider.

Referring to the attached flowcharts and other figures herein, which are described with regard to utilizing the OpenMed Web Reverse Scheduling platform or of another third party provider, e.g., HMO, healthcare insurance, etc. that incorporates the Reverse Scheduling platform described herein, the Reverse Scheduling platform can be utilized by a healthcare service provider, such as a physician, directly, or by the third-party, such as an insurance company, HMO or a hospital. In any scenario, the Reverse Scheduling platform will send a notification suggesting an appointment to a patient, preferably with a list of available times for the patient to select. In the event that the healthcare service provider is directly contacting the patient, once the patient (or several patients) selects the preferred time (or multiple times), the appointment can be immediately scheduled (assuming that another patient has not already selected the requested time) with the healthcare provider, e.g., care provider. In other implementations, if the Reverse Scheduling platform is being utilized by a third-party, once the patient selects the preferred time or times, the healthcare service provider can be contacted to either confirm that the time in question is, in fact, available, or provide alternative times.

It is noted that if the Reverse Scheduling platform is being utilized by a third-party, the patient, or even the care provider, may not be aware of who the third-party is which is sending the notification. For example, the third-party can utilize the identifying information of the healthcare provider to the patient, so that, as far as the patient is concerned, they are dealing directly with the healthcare provider, e.g., care provider.

It is noted that, in the case of a third party, information can be filtered from claims notices provided to the third-party by the healthcare service providers or other information or providers. Alternately, rather than filtering, the service provider or third party can individually and manually select one or more patients based on personal knowledge or other information known to the service provider or third party. In any case, by assisting in encouraging patients to make and keep regularly scheduled appointments, the reverse scheduling techniques taught herein, particularly with regard to the attached flowcharts and figures, can change the trajectory of medical care/expenses in the healthcare industry.

In particular, by utilizing the reverse scheduling techniques taught by the attached flowcharts and other figures, coordination can be provided between third-party healthcare companies, such as insurance companies and hospitals, providers of healthcare services, such as physicians and medical testing facilities, and patients. In short, this provides better healthcare and saves money for all concerned. As noted above, the advantages of the reverse flow appointment scheduling system and processes are not limited to the healthcare service industry, but can, instead, provide better service and save money in virtually any service industry.

It is noted that the reverse flow appointment scheduling system and processes regarding the rescheduling is particularly useful for group scheduling by filtering through a large number of patients to ensure that the notifications go only to the appropriate patients. For example, all of the patients of a provider/third-party could be filtered to determine the number of patients over the age of 50 who have not gone to a primary care physician for a selected number of years for selected procedures, e.g., colonoscopy. In this way, group scheduling can be coordinated with customized information regarding filtered groups for virtually any service.

In conjunction with this, as shown in the attached flowcharts and other figures, appropriate notifications regarding specific healthcare services can be prepared and sent to the selected filtered patients by any desired technique, including text, email, phone calls or bulk mailing. The reverse scheduling could also be set up to provide recurrent filtering of current patients so that periodic notifications can be sent which are pertinent to the current, ever-changing, patient database. In regard to this, the filtered information can be saved so that it can be applied to the current database in a recurrent manner. In other words, in the example given above, the filtering based on patients over 50 years old who have not had an appointment with a primary care physician for more than two years can be recurrently applied so that timely notices can be sent, in a real-time manner, to those patients currently in the database.

In another aspect of the reverse flow appointment scheduling system and processes, as shown in the attached flowcharts and other figures, reasons for suggesting the appointment can be provided to the patient, or, in the case of a third party, to the healthcare service provider, or both. In conjunction with this, the messages provided by a third-party to the healthcare service provider can be hidden from the patients themselves, if desired.

With regard to the healthcare providers themselves, it is noted that the Reverse Scheduling platform is intended to be utilized in conjunction with the National Provider Identifier (NPI) system, which provides a unique identification number for individual healthcare workers. This is utilized in the OpenMed Web App, for example, and is a convenient way for a third-party to coordinate the operation of the reverse scheduling process with medical service provider such as physicians and medical testing companies.

In utilizing the reverse flow appointment scheduling system and processes, is not necessary for an individual patient or service care provider to be signed up with the Reverse Scheduling platform being utilized by either the healthcare provider or a third party for implementing the reverse scheduling process.

### BRIEF DESCRIPTION OF THE DRAWINGS

The present disclosure is described in the detailed description which follows, in reference to the noted plurality of drawings by way of non-limiting examples of exemplary embodiments of the present disclosure.

FIGS. 1A-1I show a flow diagram of a system of and processes related to reverse flow appointment scheduling in accordance with aspects of the present disclosure. It should be recognized that FIGS. 1A-1I are a single flow as shown in consolidated FIG. 4, which can be implemented on an computing infrastructure or within a cloud environment as described herein.

FIG. 2 shows another flow diagram of the system and processes related to reverse flow appointment scheduling (i.e., fallout process) in accordance with aspects of the present disclosure.

FIG. 3 shows another flow diagram of the system and processes related to reverse flow appointment scheduling (i.e., appointment flow) in accordance with aspects of the present disclosure.

FIG. 4 is a consolidated flow diagram of FIGS. 1A-1I, in addition to FIGS. 2 and 3.

FIGS. 5A-5B are alternative consolidated flow diagrams in accordance with aspects of the present disclosure. These flow diagrams are different users, including a patient and service provider, e.g., health care insurer, HMO, etc.

FIGS. 6-14 are various interfaces which can be implemented with the systems and processes described herein.

FIG. 15 is a schematic computing infrastructure which is capable of implementing the steps/processes described and shown herein.

FIGS. 16-25 are interfaces which can be implemented with the systems and processes described herein for preparing and running a campaign in accordance with aspects of the present disclosure.

FIG. 26 is an interface on a user device showing a message in accordance with a campaign utilizing systems and processes described herein in accordance with aspects of the present disclosure.

### DETAILED DESCRIPTION

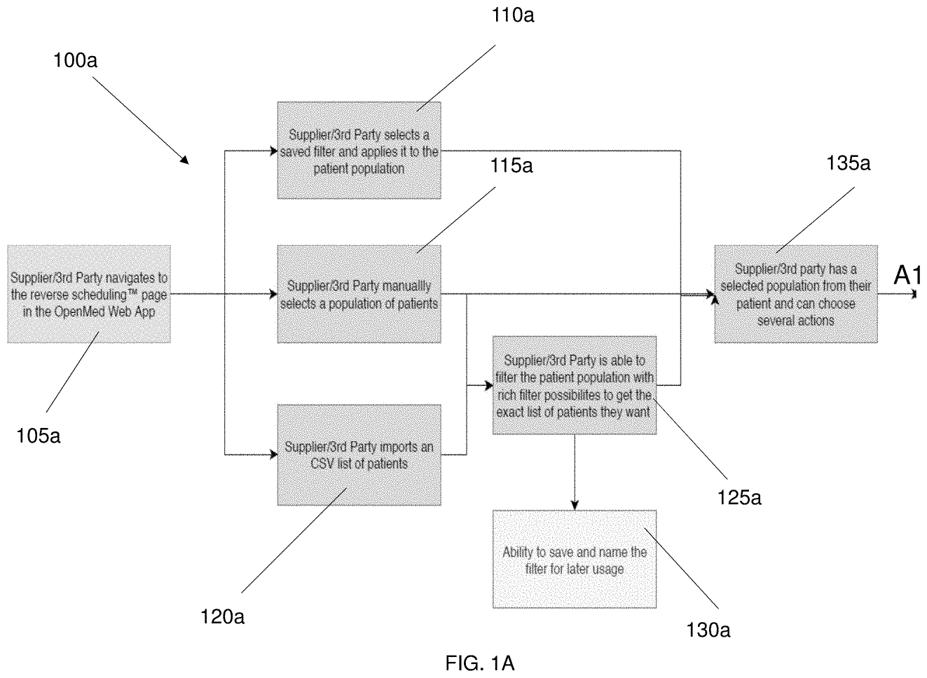

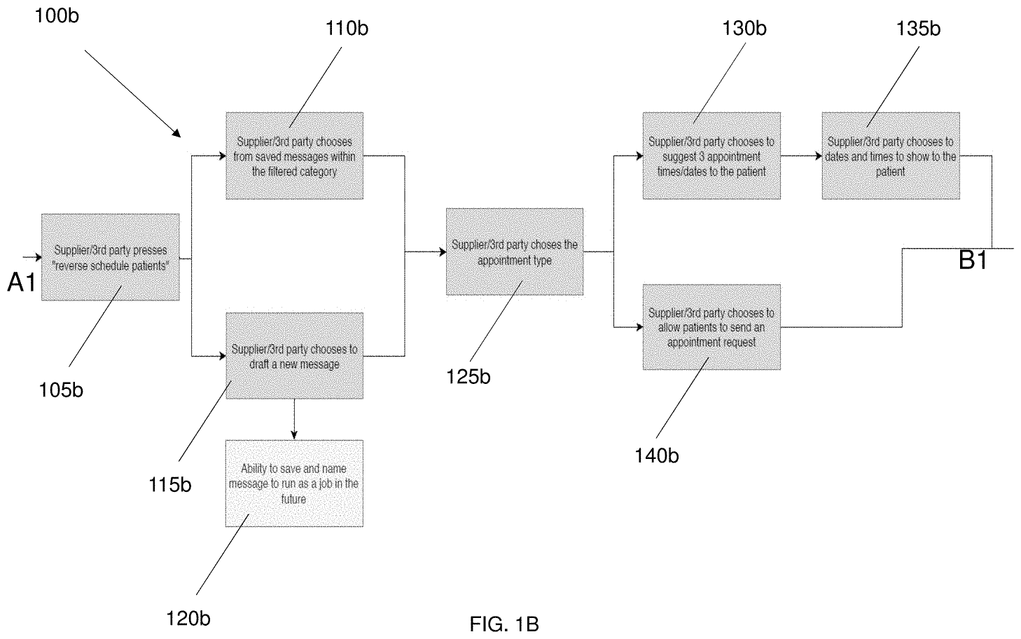

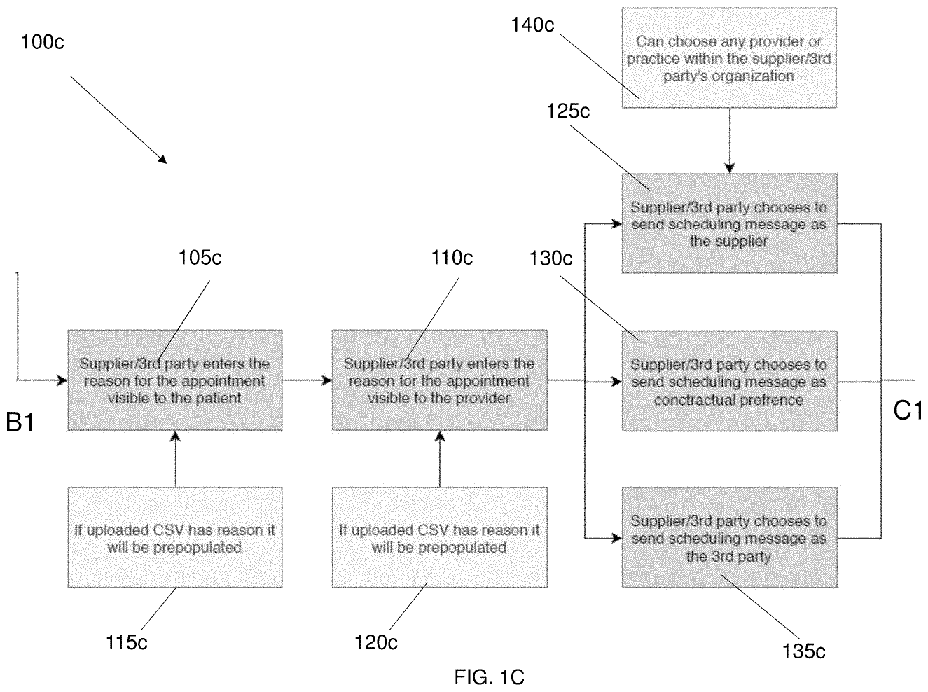

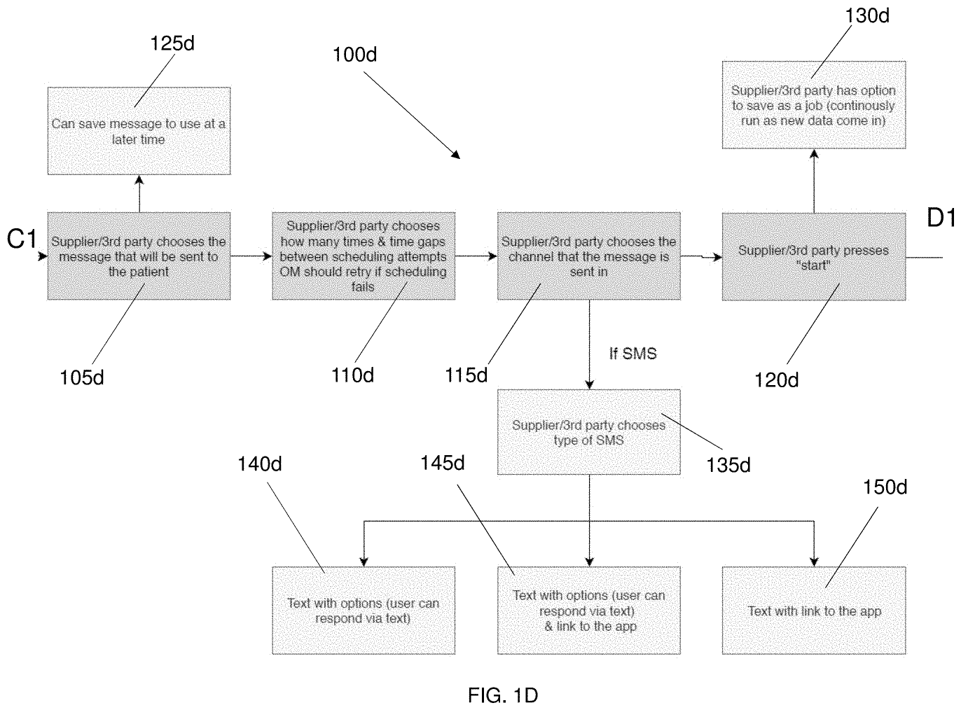

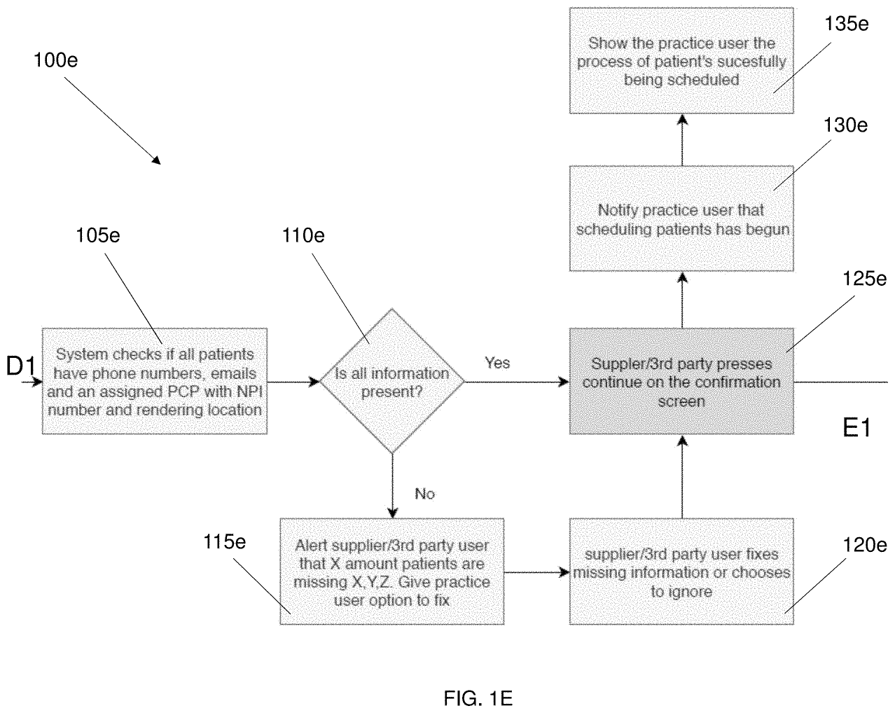

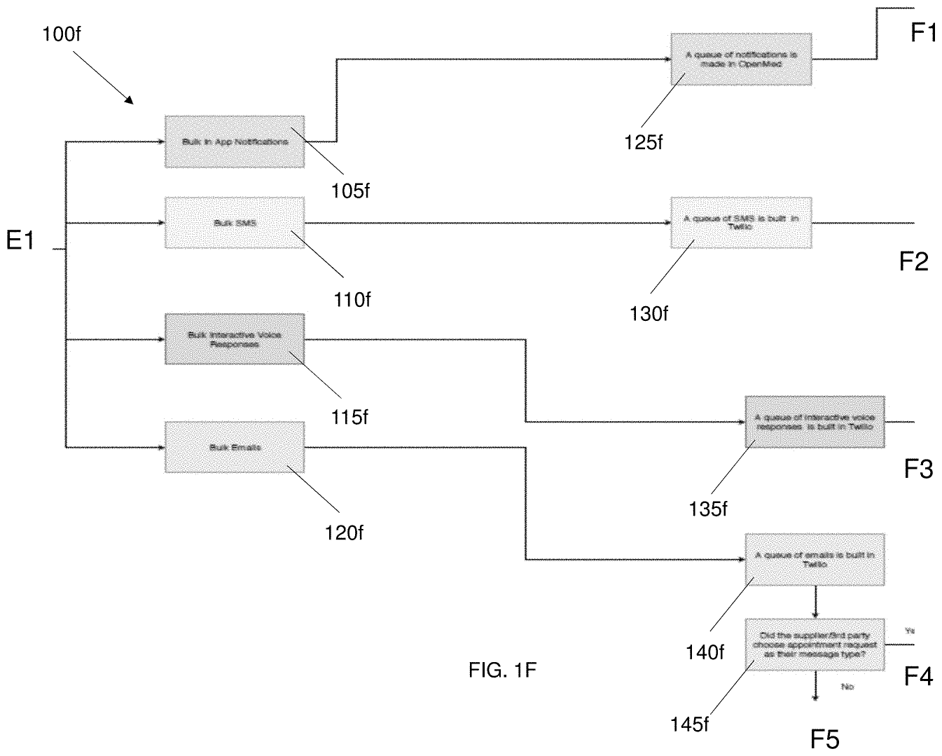

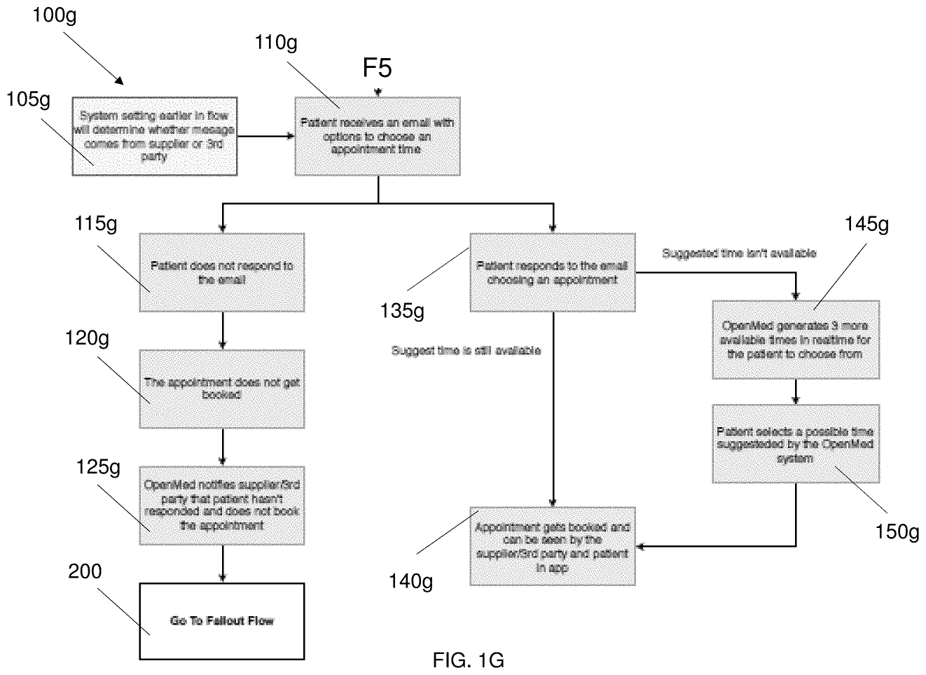

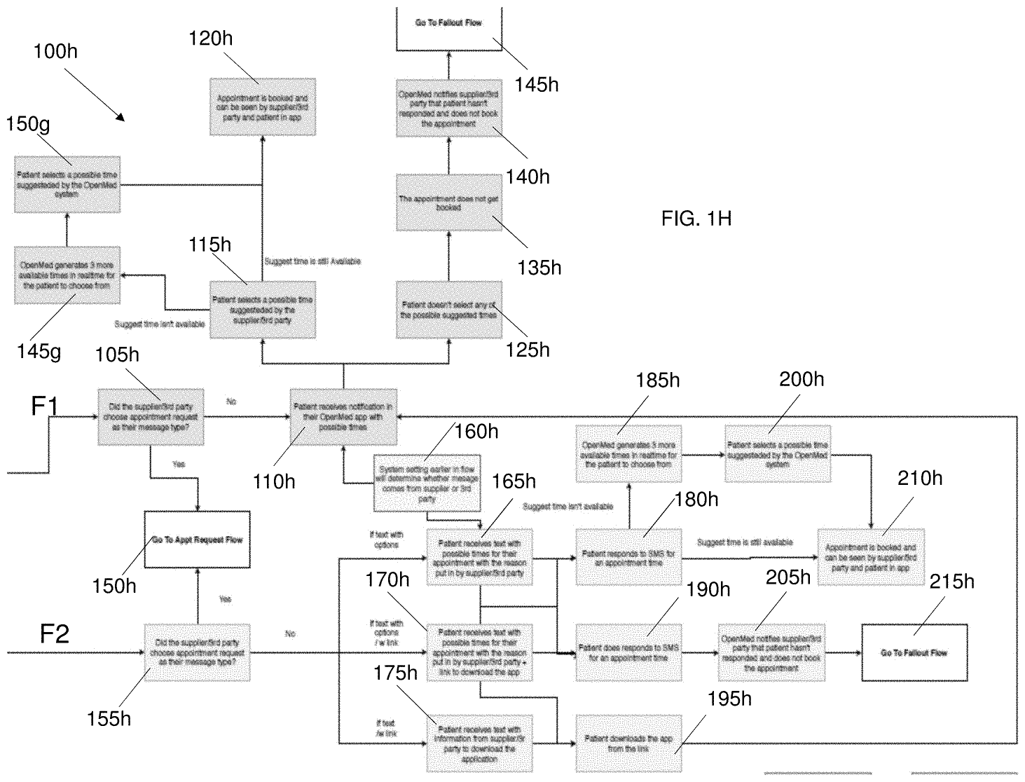

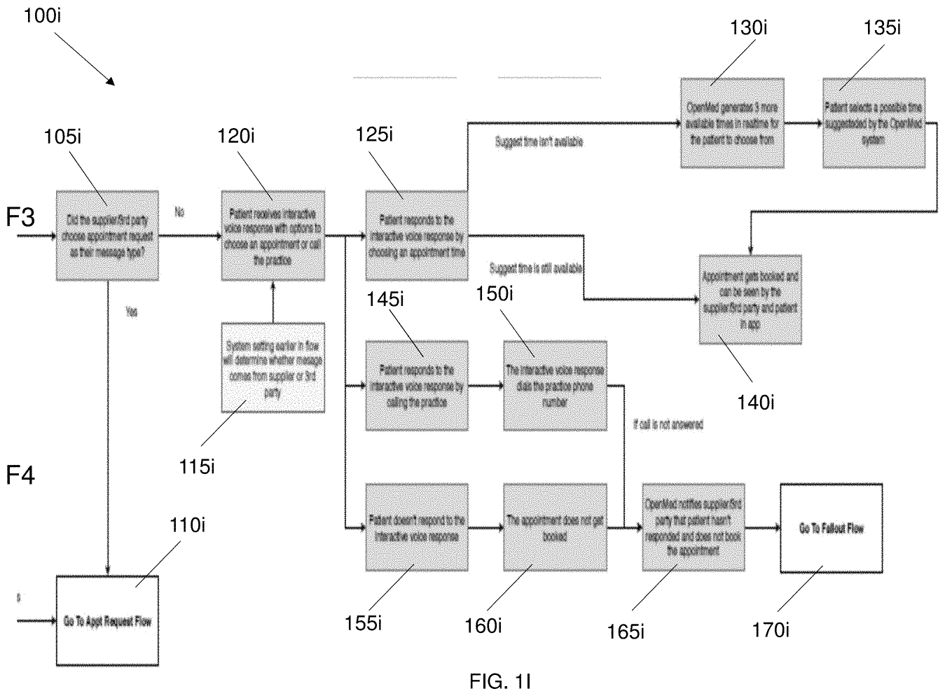

The present invention generally relates to appointment scheduling, and more particularly, to methods, systems and/or services that schedule appointments as initiated by the provider of the services. More specifically, the present invention generally relates to a methodology and systems of reverse flow appointment scheduling. The reverse flow appointment scheduling will be referred to herein as “reverse scheduling,” meaning that, instead of a consumer requesting an appointment with a service provider, the service provider, or third-party, sends a notification, for example, by text message, email, mail and/or telephone (or a multichannel notification using two or more of these modes of communication). This reverse scheduling process is explained in more detail below. It is also shown as flowcharts and schematic drawings in the attached drawing figures FIGS. 1A-26.

In embodiments, the reverse flow appointment scheduling provides an arrangement for a service provider, for example, a health plan or a medical service provider, to send notifications to the consumers recommending an appointment with the service provider, i.e., doctor, etc. In particular, as will be discussed in further detail below, and with reference to the accompanying drawings, the reverse flow appointment scheduling is particularly directed to analyzing user information regarding one or more consumers to determine if a predetermined time has passed since the one or more consumers has had an appointment with the service provider for a predetermined service. The reverse flow appointment scheduling can also base scheduling decisions on medical conditions of the consumer (patient), as well as a host of other demographic information, which can be filtered by the systems described herein.

In embodiments, gathered information regarding the consumer is filtered to limit notifications to those consumers who are coming due, or who are overdue, for an appointment or services, e.g., medical care. For example, in the medical field, this has the important practical application of preventing “care gaps,” that is, gaps in a patient's healthcare. This can be essential in the healthcare industry for discovering illnesses, or warning signs of future illnesses, early in the process which is, of course, both beneficial to patients and to the healthcare field as a whole. It is noted, however, that the reverse flow appointment scheduling has important practical applications in many fields involving service providers and consumers in ensuring that the consumers keep on a regular schedule for appointments. Just a few examples include maintenance of equipment and machinery, such as automobiles, air-conditioners, computers, copiers and printers, etc. In fact, the reverse flow appointment scheduling has applicability in virtually any field involving providing services where regular appointments, be it for healthcare or maintenance, are important.

### Flow Diagrams for Implementing the Reverse Flow Appointment Scheduling

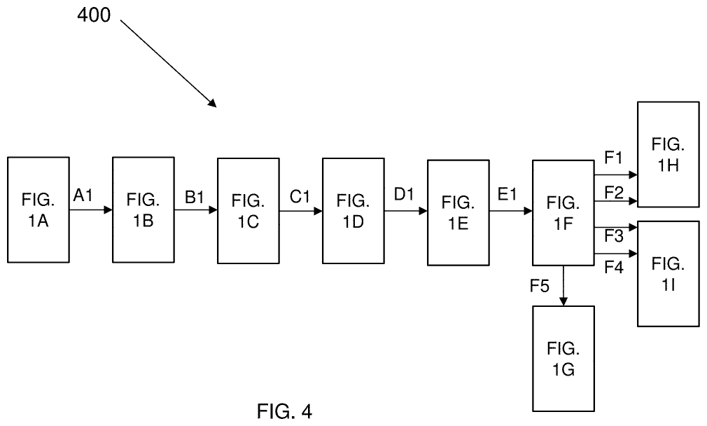

FIGS. 1A-4 show several different flow diagrams for implementing the system of and processes related to reverse flow appointment scheduling in accordance with aspects of the present disclosure. As illustrated in FIG. 4, and as should be understood by those of skill in the art, FIGS. 1A-1I, 2 and 3 can represent a single flow of the reverse scheduling process from the starting point of the provider/third-party navigating to the reverse scheduling page in the OpenMed Web Reverse Scheduling platform (shown on the left side of FIG. 1A) to the point of either going to the fallout flow or the appointment request flow (shown in FIG. 1I). For ease of understanding, though, FIGS. 1A-1I, 2 and 3 are broken into separate flows for discussion.

The flow diagrams and blocks in the Figures illustrate the architecture, functionality, and operation of possible implementations of systems, methods, and computer program products according to various embodiments of the present disclosure. In this regard, each block in the flow diagrams or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Solely for purposes of non-limiting example, the following discussion, and the FIGS. 1A-1I and 2-4 are directed to a specific example of the healthcare industry in which a healthcare provider, such as a physician, and/or a third-party, such as a health insurance company or hospital, health plan, primary or secondary provider, flow diagrams of pharmaceuticals, etc. (hereinafter referred generally as “service provider”) analyzes/filters patient data:

1. prepares a list of available appointment times which are available for a medical service provider to meet with the one or more patients to provide a predetermined service, and

2. prepares a notification to one or more patients with a list of available appointment times, and sends the notification to the one or more patients to request that the patient schedule appointments by selecting one or more of the appointment times from the list.

However, as noted above, the techniques disclosed herein could be utilized in virtually any service industry.

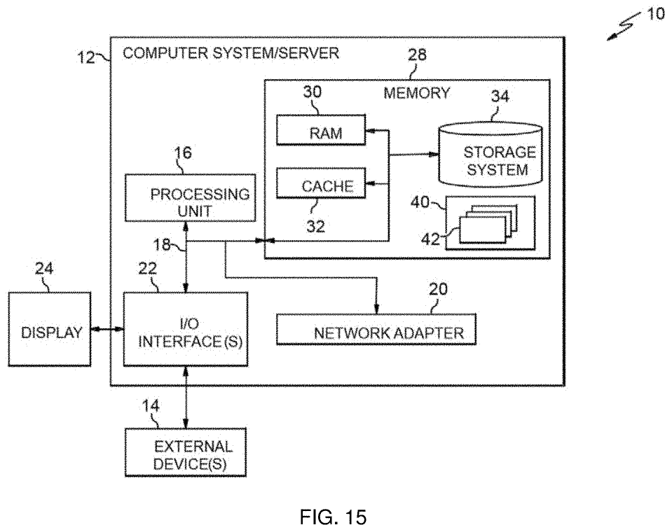

In embodiments, the service provider gathers information about the patient. This information is stored in a storage device of one or more computing infrastructures as shown in FIG. 15, for example. The patient information can include, for example, name, address, contact information (e.g., email, phone number, or other contact information), medical condition of patient, e.g., diabetic, arthritis, heart disease, etc. The information can also include medications used, dates last prescribed, doctors which have seen the patient and their pertinent information (address, specialty, appointment availability), etc.

Additional information may include last appointment with a physician or other care provider, prognosis, diagnoses and a host of other medical information. Any subset and/or all of this information can be filtered by the one or more computing infrastructures to determine any desired subset of patients, e.g., all patients that are diabetic, take certain medications and have not seen their primary care physical within the last six months. In this way, any subset of patients can be filtered for further analysis and/or auto notifications by the service provider.

Accordingly, as an example of a practical application of the reverse flow appointment scheduling, the reverse scheduling techniques disclosed in the flowcharts shown in FIGS. 1A-I and 2-5B can be used to filter all or any subset of patients of a third party or service provider with diabetes who have not had an appointment in 18 months for certain tests. The information is then used to prepare notifications to contact those diabetic patients who are uncovered through the filtering process. Another example would be filtering all patients to find and notify all congestive heart failure (CHF) patients who have not had Body/Mass Index (BMI) measurements taken within the last 10 months.

In order to provide such notifications, certain information may be required and stored, as noted above. For example, the third-party/service provider should know the first and last name of the patients, contact information, such as email address, home address and/or telephone numbers, and, preferably, the appropriate physician to provide the services in question. It is noted that, optionally, an appropriate physician can be selected for those patients who do not have an physician for the service in question. For example, a health insurance company, an HMO or a hospital (e.g., service providers) could select an appropriate physician for a diabetes patient to obtain certain tests if the patient does not already have an appropriate physician for conducting and analyzing the test in question. Similarly, a primary care physician could send a recommendation for a specialist to a patient for specialized services which the primary care physician does not normally provide.

In an exemplary illustration, the service provider can navigate to a home page of the app, e.g., OpenMed™ app, which provides the proper access to the appropriate information. This can be a standalone application, which is an enterprise system of the service provider, e.g., hospital, health insurance, HMO, etc., or a third party application such as the reverse scheduling application of OpenMed™. In any scenario, once the appropriate information is gathered and a filtering process of the information is performed, the service provider can begin the scheduling process. It should be understood that the filtering can be automated, e.g., programmed to run for certain subset of patients with certain needs, or can be manually selected for a certain subset of patients. In alternative methods, the service provider can import a CSV list of patients. In any event, in the processes described herein, the service provider can filer the patient population to any granularity that is desired, in order to obtain any subset of patients (consumers).

The system and processes herein have the ability to save and name the filtered information for later use. In this way, the filter does not need to be run multiple times for the same information. In further embodiments, as patients are added or subtracted by the system, the filter can be run at certain, predetermined intervals, to ensure that the filtered information, e.g., subset of patients, remains current.

In FIG. 1A, the flow 100a begins with step 105a, in which the Provider/3rd party navigates to the Reverse Scheduling™ page in the OpenMed Web App. At step 110a, the Provider/3rd party selects a saved filter and applies it to the patient population. Alternatively, at step 115a, the Provider/3rd party can manually select a population of patients. Further, in step 120a, the Provider/3rd party can import a comma-separated values (CSV) list of patients obtained from steps 110a and 115a.

Flow 100a continues with step 125a in which the Provider/3rd party is able to filter the patient population with further rich filter parameters to get an exact list of patients desired by the Provider/3rd party. At step 130a, the Provider/3rd party has an ability to save and name the applied filter for later usage. At step 135a, the Provider/3rd party has a selected population from their patient database and can choose several actions.

After the filtering, the provider can select the certain population and chose certain actions. These actions can include, e.g., sending a notification by a certain means such as email or text or phone, to the selected patient population. The service provider can chose from saved message within the filtered category. These messages can be sent to the patient, the care provider or saved with the service provider. The service provider can also select to draft a message, with the ability to save the message to run as a job in the future. This message may be used to program the system to run at certain times, perform certain actions or be provided to the patient or care giver as a note concerning the appointment and/or recommended treatments, etc.

Still referring to the figures, the service provider can choose to suggest appointments, e.g., three or more appointment times with certain care providers. The appointment dates and times can be prearranged with the care provider or suggested by the service provider. In one implementation, for example, the service provider may have access to the calendar of one or more of the care providers, which will provide care to any or all of the subset of patients in which a notification was sent. For example, the service provider, in the filtering process, could have identified one or more care providers that are associated with the one or more of the subset of patients, which have been identified by the filtering process.

The system can then access the care provider calendar, determine open appointment times and send a notification to the patient for such times. Alternatively, the system and processes described herein can select suggested times and dates, which would need to be approved by the care provider at the time of appointment request. In an alternative method, the service provided can choose to allow the patient to send an appointment request. In any scenario, the service provider can enter the reason for the appointment into a screen that is visible to the patient. This will provide will the patient with additional information for the need and/or urgency of an appointment. The service provider can select any provider or practice, e.g., care provider, within the service provider organization. For example, if the service provider is an insurance company, the service provider can select any care provider that has a contract with the health insurance company.

The service provider can select to send the appointment and/or scheduling message to the provider, e.g., care giver, to a contractual preference or to any other third party. This will then provide notification to the provider of services that an appointment with a particular patient is being schedule or requested to be scheduled. The message to the care provider, contractual preference, or other third party can be saved in the storage system as shown in FIG. 15, for example. The service provider can then choose to send the message or other message to the patient (and/or other third party as noted herein).

In embodiments, the service provider can select how many times and the time gap between the scheduled attempts, if there is a failure on the part of the patient to respond to the notification and/or make an appointment. The service provider can also select how many times and the time gap between the scheduled attempts based on other criteria, such as severity of a preexisting condition or other patient need or condition. If the attempts have failed, the scheduling can be retried any predetermined amount of times.

The service provider can select the means for sending of the notification, e.g., channel. This can include, for example, a type of SMS, email, phone, etc. In this embodiment, the SMS can include text with options, i.e., user can respond by text, can be directed to an app, e.g., OpenMed™ Reverse Scheduling platform or service provider app, to see the particulars of the notification and, in embodiments, make an appointment through the app, or any combination thereof.

As an example, FIG. 1B shows an exemplary flow 100b in utilizing the obtained and filtered patient information following the steps of FIG. 1A. Specifically, flow 100b continues from point A1 following step 135a of flow 100a of FIG. 1A by beginning with step 105b. In step 105b, the Provider/3rd party presses “reverse schedule patients” on a display interface (see, for example, the displayed 24 in FIG. 15) in order to begin the process of reverse scheduling patients listed in the filtered list. At step 110b, the Provider/3rd party can choose from saved messages within the filtered category. Alternatively, the Provider/3rd party can choose to draft a new message at step 115b, which allows an ability to save and name messages to run as a job in the future at step 120b.

At step 125b, the Provider/3rd party can choose the appointment type for the reverse scheduling process. This is followed by step 130b or step 140b. At step 130b, the Provider/3rd party chooses to suggest three appointment times/dates to the patient, which is followed by the Provider/3rd party choosing dates and times to show to the patient at step 135b. Alternatively, at step 140b, the Provider/3rd party can choose to allow patients to send an appointment request.

FIG. 1C illustrates an exemplary flow 100c for generating scheduling messages following point B1 in FIG. 1B. Specifically, flow 100c continues from step 135b or step 140b of flow 100b by beginning with step 105c. In step 105c, the Provider/3rd party enters the reason for the appointment visible to the patient. In embodiments, step 115c discloses that if the uploaded CSV list of patients has the reason for the appointment already listed in the CSV list, the reason will not need to be entered at step 105c. Instead, the reason will be pre-populated from step 115C.

At step 110c, the Provider/3rd party enters the reason for the appointment visible to the provider. In embodiments, similar to step 115c, if the uploaded CSV list already has the reason for the appointment, the reason for the appointment will be pre-populated in step 120c. Flow 100c continues with either step 125c, step 130c or step 135c. In step 125c, the Provider/3rd party can choose to send the scheduling message as the provider. Alternatively, at step 130c, the Provider/3rd party can choose to send the scheduling message as a contractual preference. In even further embodiments, at step 135c, the Provider/3rd party can choose to send the scheduling message as the third party. Regardless of whether step 125c, 130c or 135c is selected, step 140c allows the Provider/3rd party to choose any provider or practice within the Provider/3rd party's organization.

FIG. 1D illustrates an exemplary flow 100d for choosing the type of scheduling message following point C1 of FIG. 1C. Specifically, flow 100d continues from steps 125c, 130c or 135c of flow 100c by beginning with step 105d. In step 105d, the Provider/3rd party chooses the message that will be sent to the patient. In embodiments, this message can be saved to use at a later time at step 125d. At step 110d, the Provider/3rd party chooses how many times and the amount of time gaps between scheduling times, and whether the scheduling attempt should be retried if the original scheduling attempt fails.

Flow 100d continues with step 115d, in which the Provider/3rd party chooses the channel that the message is sent in. In embodiments, if the message is sent as a short message service (SMS) message, the flow 100d continues with step 135d. At step 135d, the Provider/3rd party chooses the type of SMS message.

In embodiments, the Provider/3rd party can select steps 140d, 145d or 150d. At step 140d, the text with options (user can respond via text) is provided. At step 145d, the text with options (user can respond via text) and link to the Reverse Scheduling platform (e.g., OpenMed™ or other third-party app) is provided At step 150d. Text with a link to the Reverse Scheduling platform is provided without providing for direct response to the user. Regardless of the selection of steps 140d, 145d or 150d, flow 100d continues with step 120d. At step 120d, the Provider/3rd party presses “start” on the display interface in the OpenMed™ Reverse Scheduling platform or other third-party app.

In embodiments, at the start of the session, the system and processes described herein can check to see that all patient information is available. This information can be the phone numbers, email address an assigned PCP (primary care physician) with NPI number and rendering location, etc. If any or all of the information is not available, an alert can be sent to the service provider, at which time the service provider can request additional information or otherwise attend (fix) to the issue. A third party can fix the missing information or choose to ignore that there is missing information. As an example, FIG. 1E shows an exemplary flow 100e in confirming patient information following point D1 of FIG. 1D. Specifically, flow 100e continues from step 120d of flow 100d by beginning with step 105e. In step 105e, the system checks if all patients have phone numbers, emails and an assigned primary care provider (PCP) with a national provider identifier (NPI) number and a rendering location. At step 110e, if all the information is present, the flow 100e moves onto step 125e. Alternatively, if all the information is not present, the flow 100e moves onto step 115e.

At step 115e, the Provider/3rd party is alerted that “X” amount of patients are missing information “X, Y, Z.” In embodiments, there is an option for the user to resolve the missing information. Specifically, step 120e allows for the Provider/3rd party user to fix the missing information or to ignore the missing information. Flow 100e continues with step 125e, in which the Provider/3rd party presses continue on the confirmation screen of the display interface. This is followed with step 130e, in which the practice user is notified that scheduling patients has begun. Additionally, step 135e allows for the practice user to show that one or more of the patients have been successfully scheduled for an appointment.

If all of the appropriate information is available or the third party chooses to ignore the issue, the service provider can continue with the with a confirmation screen. The system can automatically notify the practice user, e.g., care giver, that a scheduling process has begun. The system can also show the service provider or practice user the process of the patient successfully being scheduled.

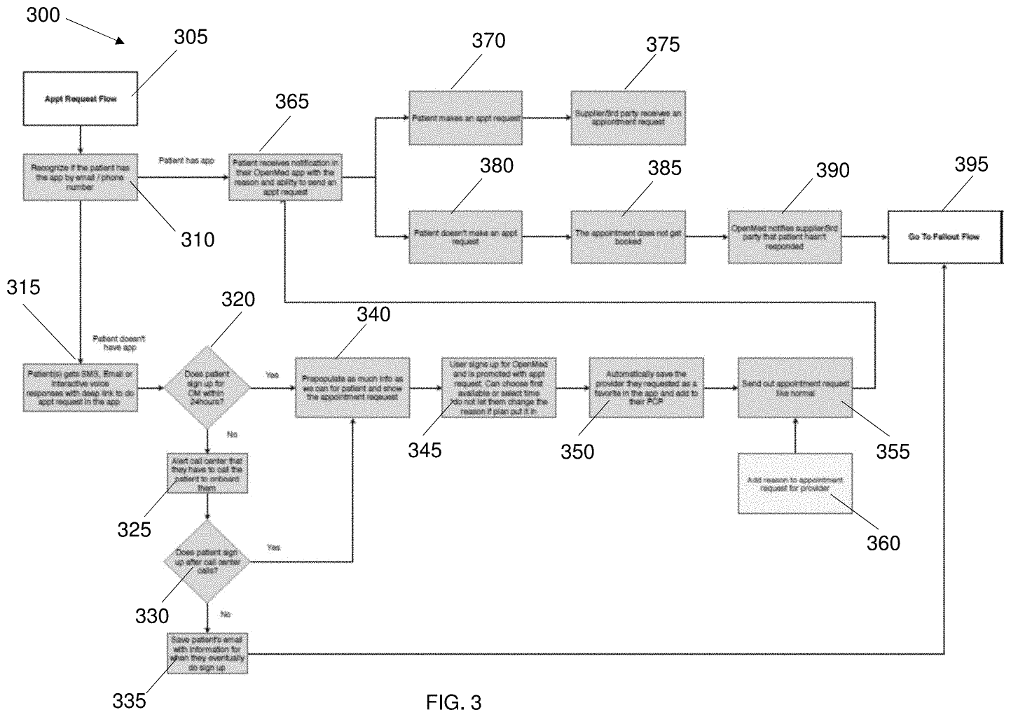

The scheduling can be performed through many different avenues, whether by bulk in the application notifications, bulk SMS, bulk interactive voice responses or bulk emails to name a few. In the bulk application notification, the flow will determine whether the service provider selected an appointment request as their message type. If so, the system and processes will go to the application request flow shown in FIG. 3, for example. This same process can be used for the bulk SMS process, where the a queue of the SMS can be built into Twilio™ or other known and appropriate API as should be understood by those of skill in the art. Reverting back to the bulk notifications in the Reverse Scheduling platform in which the service provider (or third party) does not select appointment request as their message type, the system and processes will determine whether the patient receives notification in their OpenMed™ Reverse Scheduling platform (or other app) with possible times. The patient can select a possible time suggested by the service provider and, if available, the appointment is booked and can be seen by the care provider and/or other third party, and/or the patient. If the times are not available, the Reverse Scheduling platform can then generate additional times, e.g., three times and date, in real-time for the patent to select. The patient can then select the possible suggested time, which thereafter the appointment is booked and the appointment can be seen by the care provider or other third party, and the patient. If the patient does not select any suggested times, the appointment does not get booked and the app, e.g., OpenMed™ Reverse Scheduling platform or other third party Reverse Scheduling platform using the system and processes herein, will notify the service provider, care giver or other third party that the patient has not responded and did not book any appointment. It should be understood that in the flows, the term service provider, third party and/or care giver can be interchangeably used, depending on the particular circumstances. If no appointment is booked, the processes will continue to the fallout flow.

As an example, FIG. 1F illustrates a flow 100f for bulk messaging following point E1 of FIG. 1E. Specifically, flow 100f continues from step 125e of flow 100e by beginning with step 105f. In step 105f, the bulk messages can be in the OpenMed™ or other third-party Reverse Scheduling platform notifications. Alternatively, at step 110f, the bulk messages can be bulk SMS. In further embodiments, at step 115f, the bulk messages can be bulk interactive voice responses. In even further embodiments, the bulk messages can be bulk emails at step 120f.

Depending on the step selected from steps 105f, 110f, 115f or 120f, the flow 100f will proceed with steps 125f, 130f, 135f or 140f. If step 105f is selected, flow 100f goes to step 125f. At step 125f, a queue of notifications is made in the OpenMed™ or other third-party app. If step 110f is selected, flow 100f goes to step 130f. At step 130f, a queue of SMS is built in Twilio, for example, or another cloud communication package. If step 115f is selected, flow 100f goes to step 135f. At step 135f, a queue of interactive voice responses is built in Twilio, for example, or another cloud communication package. If step 120f is selected, flow 100f goes to step 140f. At step 140f, a queue of emails is built in Twilio, for example, or another cloud communication package. This is followed by step 145f, in which the question is asked on whether the Provider/3rd party can chose the appointment request as their message type.

Reverting back to the bulk SMS situation, when the service provider, caregiver and/or third party does not select an appointment request as their message type, the system and processes will continue to one of three processes, for example:

1. Patient receives text with possible times for their appointment with the reason put in by provider/3rd party.

2. Patient receives text with possible times for their appointment with the reason put in by provider/3rd party+link to download the Reverse Scheduling platform.

3. Patient receives text with information from provider/3^{rd} party to download the application.

In situation #1, the patient can respond to SMS for an appointment time. If a suggested time is not available, the system and processes will generate more available times in real-time for the patient to choose from. This can be done by interfacing with the care giver's calendar, for example. The patient can then select a suggested time using the OpenMed™ or other third party app, as noted herein. Similar to the bulk Reverse Scheduling platform situation, the appointment is booked and it can be seen in the app. If the patient responds initially to the appointment time, then the appointment is booked and it can be seen in the app.

In situation #2, the patient may not respond to SMS for an appointment time, the OpenMed™ Reverse Scheduling platform or other third party Reverse Scheduling platform notifies the provider/3^{rd} party that the patient has not responded and does not book the appoint. In this case, the flow will continue with the fallout flow.

In situation #3, the patient can download a link to schedule an appointment (and provide any additional information including patient information and messages). The process flow will revert back to the processes of the bulk Reverse Scheduling platform situation starting with, for example, the patient receiving the notification in their OpenMed™ or other third party app.

Referring back to the situation where the Bulk Interactive Voice Provider/3rd party chooses responses, the process will process as follows:

1. A queue of interactive voice responses is built in Twilio or other API.

2. The flow determines whether the provider/3rd party selected an appointment request as their message type? If so, the process continues with the appointment request flow. If not, the patient receives an interactive voice response with options to choose an appointment or call the practice.

3. The patient responds to the interactive voice response by choosing an appointment time. If the time is available, then the appointment gets booked and can be seen by the provider/3rd party and/or patient in the app. If an appointment is not available, additional times can be generated and the patient can then select from the additional times. If the patient selects the additional time, the appointment gets booked and can be seen by the provider/3rd party and/or patient in app.

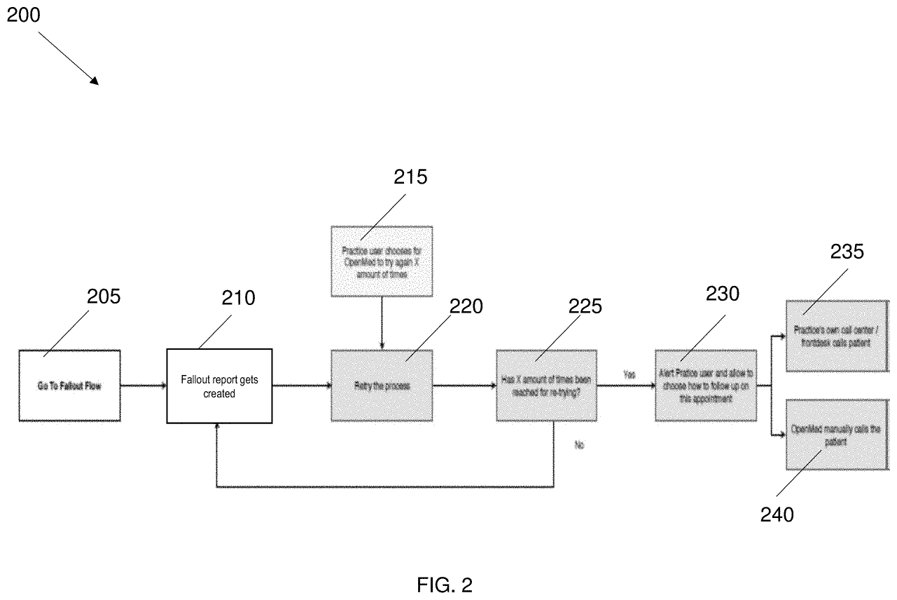

In further embodiments, the patient can respond to the interactive voice response by calling the practice of the care giver, for example. For example, the interactive voice response can automatically dial the practice phone number. The OpenMed™ or other third party Reverse Scheduling platform as described herein notifies the provider/3^{rd} party that patient has not responded and has not booked the appointment. The process will then go to the fallout flow of FIG. 2. Should the patient not respond to the interactive voice response, the appointment does not get booked. The OpenMed™ Reverse Scheduling platform or other third party Reverse Scheduling platform as described herein notifies the provider/3^{rd} party that patient has not responded and does not book the appointment. The process will then go to the fallout flow of FIG. 2.

Reverting now to the bulk email situation, a queue of emails is built in Twilio or other appropriate API. A determination is made as to whether the provider/3rd party selected an appointment request as their message type. If yes, the system and processes will continue with the appointment request flow in FIG. 3. If the patient did not receive an email with options to choose an appointment time, the appointment will not get booked, and the OpenMed™ Reverse Scheduling platform or other third party Reverse Scheduling platform notifies the provider/3^{rd} party that patient has not responded and does not book the appointment. The processes will then continue with the fallout flow of FIG. 2.

As an example, FIG. 1G illustrates a flow 100g for a patient responding to the message following point F5 of FIG. 1. Specifically, flow 100g begins with step 105g, in which the system setting earlier in the scheduling flows of flows 100a, 100b, 100c, 100d, 100e or 100f, will determine whether the message comes from the provider or the third party. In step 110g, following step 145 of FIG. 1F, the patient receives an email with options to choose an appointment time. If the patient does not respond to the email at step 115g, flow 100g proceeds to step 120g. At step 120g, the appointment does not get booked. The flow 100g then proceeds to step 125g, in which the OpenMed™ Reverse Scheduling platform notifies the provider/3^{rd} party that the patient has not responded, and does not book the appointment. This is followed by process flow 200, which is the fallout flow of FIG. 2.

Alternatively, if the patient does respond to the email by choosing an appointment at step 135g, the process flow 100g goes to step 140g. At step 140g, the appointment gets booked and can be seen by the provider/3^{rd} party and patient in the OpenMed™ app. Further, step 145g occurs after the completion of step 135g, and includes the OpenMed™ Reverse Scheduling platform generating three more available times in real-time for the patient to choose from for an appointment. At step 150g, the patient selects a possible time suggested by the OpenMed™ system.

FIG. 1H illustrates a flow 100h for selecting an appointment following points F1 and F2 of FIG. 1F. At step 105h, following point F1 of FIG. 1F, the provider/3rd party chooses an appointment request as their message type. At step 110h, the patient receives notification in their OpenMed™ Reverse Scheduling platform with possible times. In embodiments, at step 115h, the patient selects a possible time suggested by the provider/3rd party. If the suggested time is available, flow 100h books the appointment at step 120h, which can be seen by the provider/3rd party and patient in the OpenMed™ app.

Alternatively, if the suggested time is not available, the flow goes back to step 145g of flow 100g, where the OpenMed™ Reverse Scheduling platform generates 3 more available times in real-time for the patient to choose from. At step 150g of flow 100g, the patient selects a possible time suggested by the OpenMed™ system and, at step 120h, an appointment is booked.

Alternatively, if the patient does not select any of the possible suggested times at step 125h, flow 100h goes to step 135h, in which the appointment does not get booked. At step 140h, the OpenMed™ Reverse Scheduling platform notifies the provider/3rd party that the patient has not responded and does not book the appointment. Flow 100h then goes to the Fallout Flow illustrated in FIG. 2. However, if step 105h is satisfied, flow 100h goes onto step 150h, which goes to the appointment request flow, shown, for example, in FIG. 3.

As an alternative flow, following point F2 of FIG. 1F, flow 100h can begin with the provider/3rd party choosing the appointment request as their message type at step 155h. If “yes,” process 100h goes onto step 150h, which goes to the appointment request flow shown, for example, in FIG. 3. If “no,” process 100h goes to steps 165h, 170h or 175h. In embodiments, if text with options is selected, the flow 100h goes to step 165h, in which the patient receives texts with possible times for their appointment with the reason put in by the provider/3rd party.

At step 180h, the patient responds to the SMS message for an appointment time. If the suggested time in the SMS message is not available, flow 100h goes to step 185h, in which the OpenMed™ Reverse Scheduling platform generates 3 more available times in real-time for the patient to choose from. At step 200h, the patient selects a possible time suggested by the OpenMed™ system, and ends with step 210h, in which the appointment is booked, which can be seen by the provider/3rd party and patient in the app.

Alternatively, if text with options with a link is selected, flow 100h goes to step 170h, in which the patient receives text with possible times for their appointment with the reason put in by the provider/3rd party, plus the link to download the app. Flow 100h then continues with step 190h, in which the patient does responds to the SMS message for an appointment time. At step 205h, the OpenMed™ Reverse Scheduling platform notifies the provider/3rd party that the patient has not responded, and does not book the appointment. This is followed by step 215h, in which processes of the present disclosure go to the Fallout Flow shown in FIG. 2. In further embodiments, if text with a link is selected, flow 100h goes to step 175h, in which the patient receives a text with information from a provider/3rd party to download the application. Flow 100h then concludes with step 195h, in which the patient downloads the OpenMed™ Reverse Scheduling platform from the link.

FIG. 1I illustrates a flow 100i for patient interactions with a voice response, following points F3 and F4 of FIG. 1F. From point F3 of FIG. 1F, flow 100i begins with step 105i, in which the provider/3rd party chooses an appointment request as their message type. If “yes,” flow 100i goes to step 100i, from which flow 100i goes to the appointment request flow shown in FIG. 3. Similarly, from point F4 of FIG. 1F, if the answer is “yes” in step 145f of FIG. 1F, the flow will go to step 110i of FIG. 1I, to go to the appointment request flow of FIG. 3. Alternatively, is the answer is “no,” flow 100i goes to step 120i, in which the patient receives the interactive voice response with options to choose an appointment or call the practice. In embodiments, step 115i indicates that the system setting in the earlier flows 100a, 100b, 100c, 100d, 100e, 100f, 100g and 100h will determine whether the message comes from the provider or a 3rd party.

Flow 100i continues with step 125i, 145i, 155i. Step 125i is undertaken if the patient responds to the interactive voice response by choosing an appointment time. If the suggested time is not available, flow 100i goes onto step 130i, in which the OpenMed™ Reverse Scheduling platform generates 3 more available times in real-time for the patient to choose from. At step 135i, the patient selects a possible time suggested by the OpenMed™ system.

Alternatively, at step 145i, the patient responds to the interactive voice response by calling the practice. Flow 100i goes onto step 150i, in which the interactive voice response dials the practice phone number. If the call is not answered, flow 100i goes onto step 165i, in which the OpenMed™ system notifies the provider/3rd party that the patient has not responded, and does not book the appointment. The process 100i concludes with going to the Fallout Flow, shown in FIG. 2, at step 170i.

Should the patient respond to the email choosing an appointment for a specific time, the appointment gets booked and can be seen by the provider/3rd party and/or patient in the app. If the suggested time is not available, the app, e.g., OpenMed™ or other third party app, generates more available times in real-time for the patient to choose from. The patient can then select a possible new suggested time by the Reverse Scheduling platform and it get booked. The OpenMed™ Reverse Scheduling platform or other third party Reverse Scheduling platform notifies provider/3^{rd} party that patient has not responded and does not book the appointment. The process will then continue with the fallout flow of FIG. 2.

FIG. 2 shows the fallout flow which takes place when a patient does not respond to the notification (or multiple notifications). In the fallout flow, the fallout report can get created. The process can be retried and a determination can be made as to whether X amount of times have been reached for re-trying the scheduling of the appointment. If X amount of times has been reached, the fallout report is created (a report indicated that the patient is not scheduled). If the X time is not reached, then the system can alert the practice user and allow the practice user (e.g., care provider) to choose how to follow up on this appointment. The practice user can use their own resource, e.g., call center, or revert to the Reverse Scheduling platform as described herein.

More specifically, the fallout flow 200 shown in FIG. 2 includes the following steps following step 205 (“Go to fallout flow”) from step 215h of FIG. 1H and step 170i of FIG. 1I. At step 210, the fallout report is created. At step 215, the practice user chooses for OpenMed to try again “X” amount of times. At step 220, the process is retried. At step 225, it is determined whether X amount of times has been reached for re-trying. At step 230, the practice user is alerted to allow the practice owner to choose how to follow up on this appointment. At step 235, the practice's own cell center/front desk can be used to call the patient. At step 240, the application, e.g., “OpenMed, can manually call the patient.

FIG. 3 shows the process for a patient to reply to a notification with a request to schedule an appointment, or failure to complete scheduling an appointment, in which case the process reverts to the fallout flow of FIG. 2. The appointment request flow starts by recognizing if the patient has the Reverse Scheduling platform by email/phone. If the patient has the app, the patient receives a notification in their OpenMed™ Reverse Scheduling platform or other third party Reverse Scheduling platform (which uses the systems and processes herein) with the reason and ability to send. The patient makes an appointment request and the service provider/3rd party receives an appointment request. If the patient does not make an appointment request, then the appointment does not get booked and OpenMed™ or other third party (service provider) notifies provider/3^{rd} party that patient has not responded.

Should the patient(s) get an SMS, Email or interactive voice response with deep link to provide an appointment request in the app, a decision is made as to whether the patient has signed up for the service within a predetermined time period, e.g., 24 hours. If not, an alert call center can call the patient to onboard them. A determination is also made as to whether the patient signed up after call center calls. If not, the patient's email with information for when they eventually do sign up is saved for future reference and injection into the system.

If the patient signed up, the system and processes will prepopulate as much info as we can for patient and show the appointment request. The user signs up for OpenMed or other third party Reverse Scheduling platform and is promoted with appointment request. The patient can choose first available or select time *do not let them change the reason if plan put it in. The process can automatically save the provider they requested as a favorite in the Reverse Scheduling platform and add and then send out the appointment. The processes can add reason to appointment request for the provider.

More specifically, the appointment request flow 300 of FIG. 3 includes the following steps following the step 305 (“Go to appointment request flow”) from step 150h of FIG. 1H and step 110i of FIG. 1I. At step 310, it is determined that if the patient has the Reverse Scheduling platform by email/phone number. At step 315, the patient receives SMS, email or interactive voice responses with deep link to perform the appointment request in the app, if it is determined in step 310 that the patient does not have the app. At step 320, it is determined whether the patient has signed-up for appointment within 24 hours. If not, at step 325, an alert is provided to a call center so that they can call the patient to onboard them. At step 330, a determination is made as to whether the patient signed-up after the call center calls. At step 335, if the patient has not signed up, the patient's email with information will be saved for when they eventually do sign up. If at step 330 or step 320, the answer is yes, at step 340, the system and processes will pre-populate as much information as possible and show the appointment request. At step 345, the user signs up for OpenMed and is prompted with appointment request. The patient can choose the first available time or select a time. The patient cannot change the reason for the appointment. At step 350, the information is automatically saved, i.e., the provider they requested as a favorite, in the Reverse Scheduling platform and it can also be added to their PCP. At step 355, the appointment request is then sent. At optional step 360, a reason for the appointment request can be added for provider.

The above described steps 315-360 are carried out if the result of step 310 is a determination that the patient does not have the app. After step 355, the flow 300 will proceed to step 365 at which time the patient receives notification in their OpenMed Reverse Scheduling platform with the reason for the appointment and also provides the ability to send an appointment request. On the other hand, if the result of step 310 is a determination that the patient has the app, the flow 300 will proceed directly from step 310 to 365, as shown in FIG. 3.

If the patient makes an appointment request at step 370, at step 375 the Provider/3^{rd} party will receive an appointment request. If the patient does not make an appointment request, at step 380, then at step 385, the appointment does not get booked. At step 390, the OpenMed Reverse Scheduling platform notifies provider/3^{rd} party that patient has not responded and, at step 395, the process proceeds to the fallout flow of FIG. 2. It can also be seen from FIG. 3 that step 395 will occur following step 335 when the result of step 330 is that the patient has not signed up after call center calls.

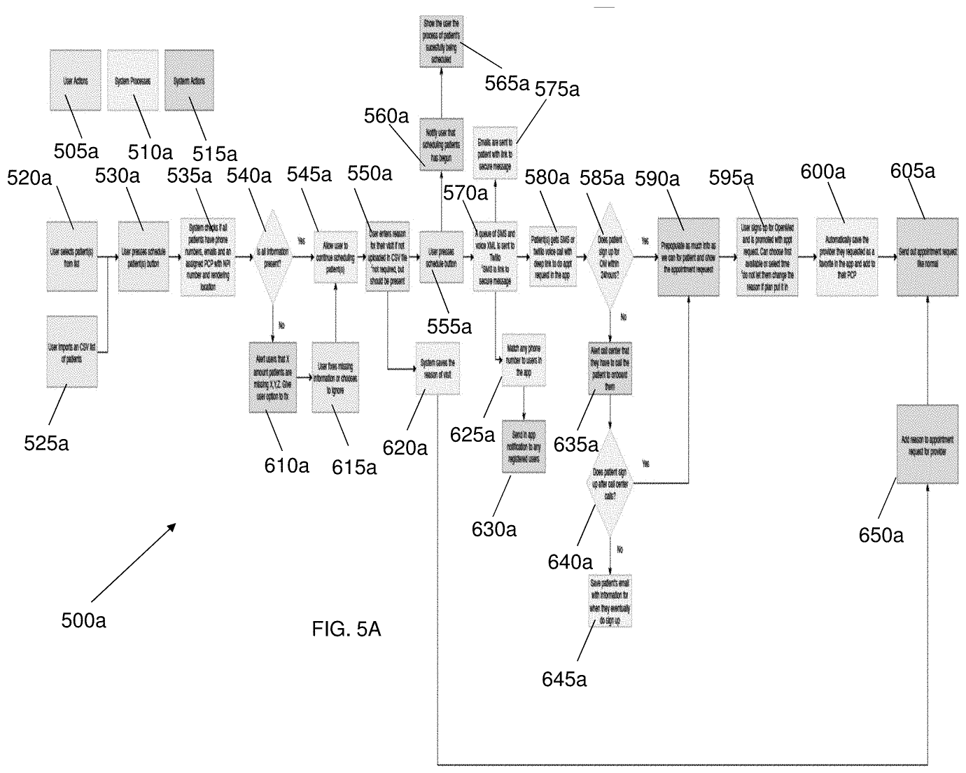

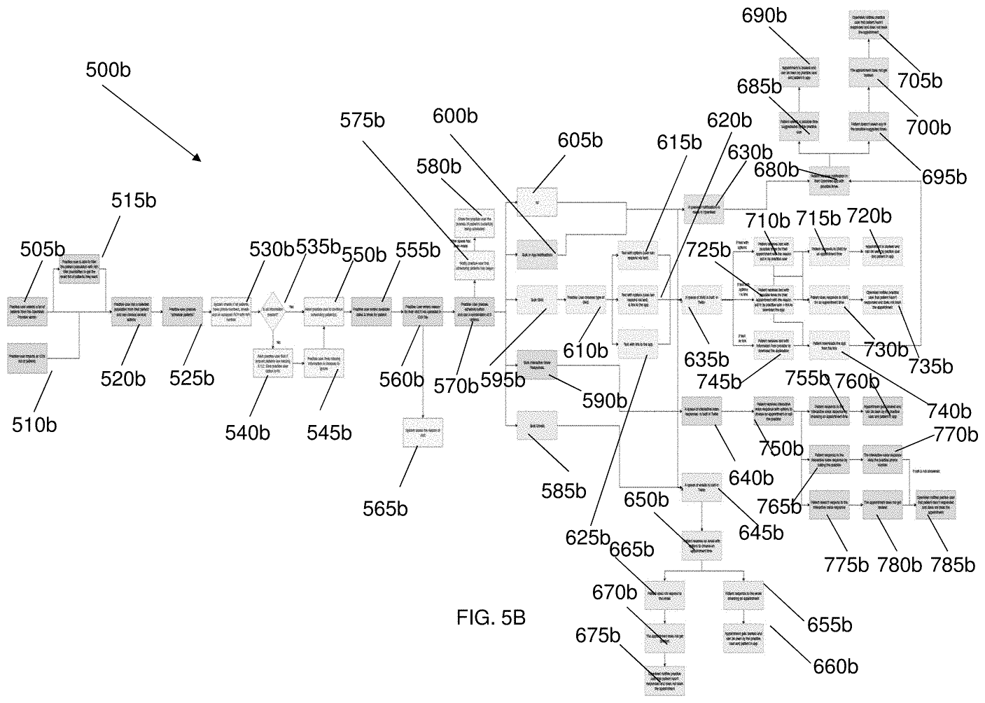

FIGS. 5A and 5B are alternative flow diagrams in accordance with aspects of the present disclosure. These flow diagrams are from a perspective of different users, including a patient and service provider, e.g., health care insurer, HMO, etc. The steps of FIGS. 5A-5B have many similarities to that shown in FIG. 4 (i.e., the compilation of FIGS. 1A-1I), for example. Accordingly, a complete description of FIGS. 5A and 5B can be seen from the figures, themselves. It should be understood, also, that the steps shown in FIGS. 5A and 5B can be injected into the flow 400 of FIG. 4 without departing from the spirit of the invention.

More specifically, FIG. 5A shows an alternative flow 500a starting from the steps 520a, wherein a user selects patients from a list. At step 525a, a user imports a CSV list of patients, and at a final step 605a, the appointment request is sent out, with a variety of alternative steps being taken along the way depending on particular situations. The steps in the flow 500a are carried out by a combination of user actions 505a, system processes 510a and system actions 515a, as shown in FIG. 5A, and as described below.

In particular, following the steps 520a and 525a, the following steps take place. At step 530a, a user presses schedule patients button and, at step 535a, the system checks if all patients have phone numbers, emails and an assigned PCP with NPI number and rendering location. At step 540a, a determination is made as to whether all information present.

If the answer to step 540a is “Yes,” the alternate flow 500a proceeds as follows. At step 545a, the system allows the continuing of scheduling patients. At step 550a, a user enters a reason for their visit if not updated in the CSV file (not required but should be present). At step 555a, a user presses the schedule button and, at step 560a, a notification is provided to the user that scheduling patients has begun. At step 565a, the system shows the user which patients have successfully being scheduled. At step 570a, a queue of SMS and voice XML is sent to Twilio SMS, for example, as linked to a secure message. At step 575a, emails or other notifications are sent to patient a with link to secure message. At step 580a, patients will receive the SMS or Twilio voice call or other message with a deep link to perform the appointment request in the app. At step 585a, a determination is made as to whether the patient has signed up for the appointment within a certain amount of time, e.g., within 24 hours.

### Interfaces for Implementing the Reverse Flow Scheduling

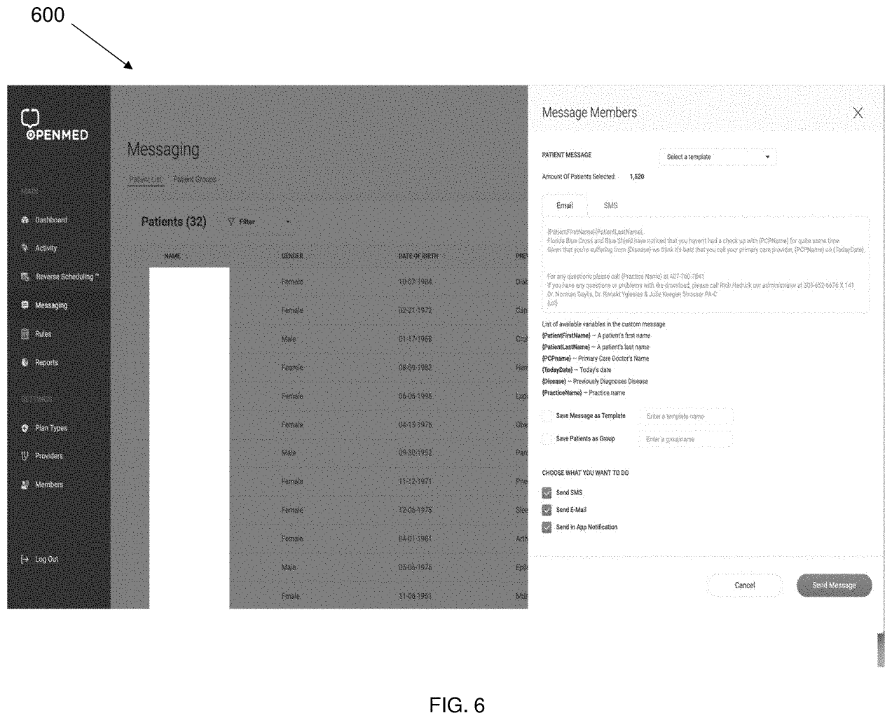

FIGS. 6-14 and 16-26 are various interfaces which can be implemented with the systems and processes described herein. For example, FIGS. 6-14 and 16-25 are interfaces which can be implemented with the systems and processes described herein for analyzing data to provide reverse flow scheduling and related processes in accordance with aspects of the present disclosure. The interfaces shown in FIGS. 6-14 and 16-26 are exemplary in nature and can be implemented in a mobile Reverse Scheduling platform or other computing structure. The interfaces can be implemented into the infrastructure shown in FIG. 15. It should also be understood that the interfaces shown in FIGS. 1-14 and 16-25 can be dynamically changed and upgraded based on input from the user.

By way of explanation, FIG. 6 is an interface 600 which can be used or has already been used to filter patient information. The patient information can include names, gender, birth dates, medical conditions, etc. The interface 600 of FIG. 6 also includes email and/or SMS messages, as well as a list of variables to provide in a custom message. The variables can include names, primary care providers, practice type of the primary care provider, date, disease or other medical conditions. The interface can be sent by SMS, email or in Reverse Scheduling platform notifications, for example.

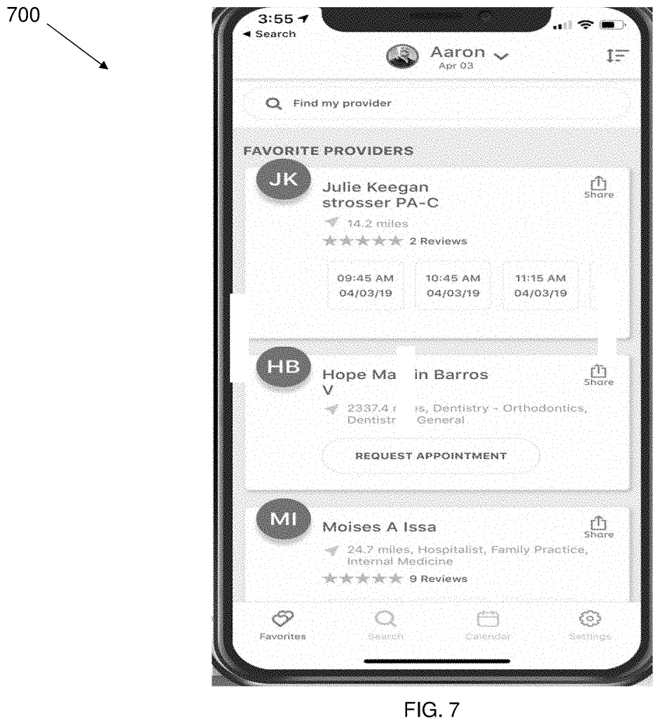

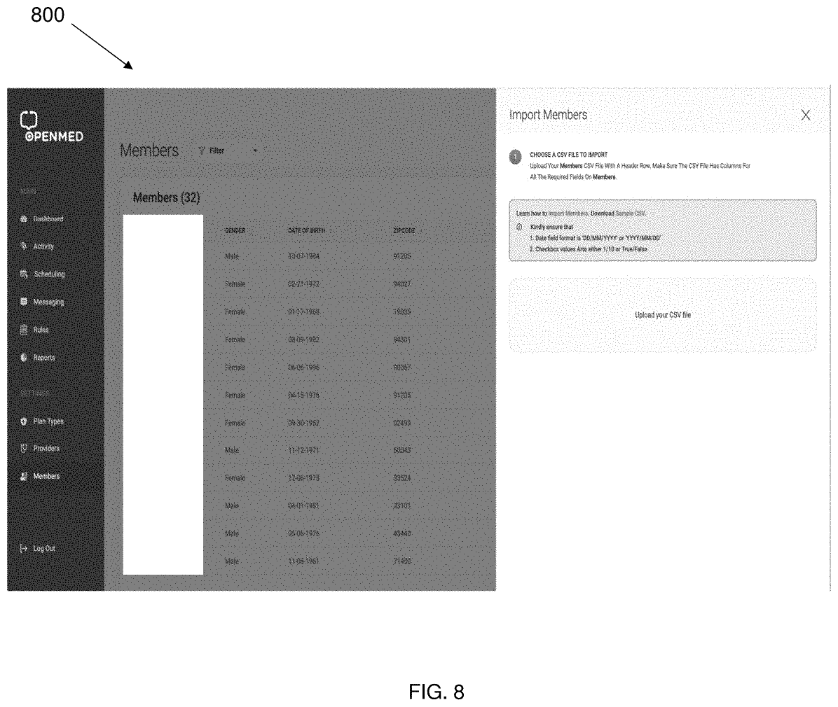

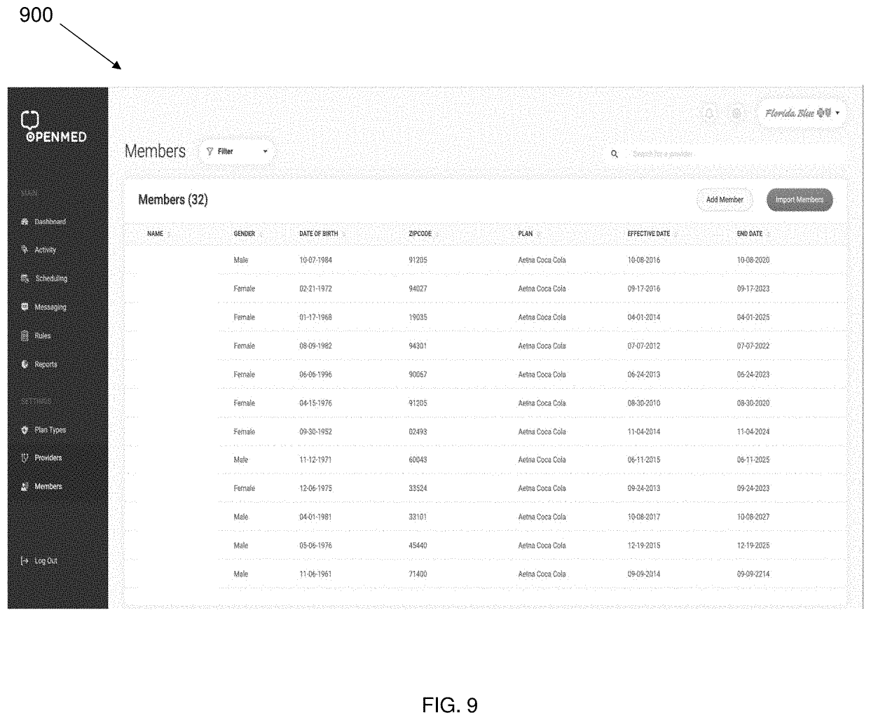

FIG. 7 is an interface 700 showing providers for the patient. By selecting the provider, it is possible to schedule an appointment using the systems and processes described herein. FIG. 8 shows information interface 800 related to members of the community, e.g., patients, providers that are subscribed to the services. FIG. 9 shows another interface 900, which in this case includes insurance company information, e.g., name of company, effective date of plan, end date of the plan, etc.

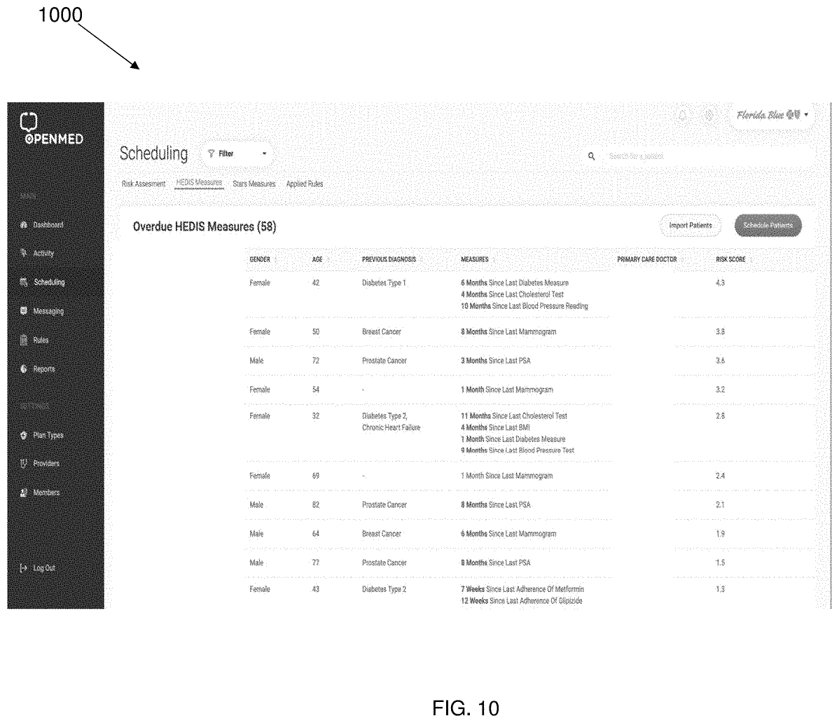

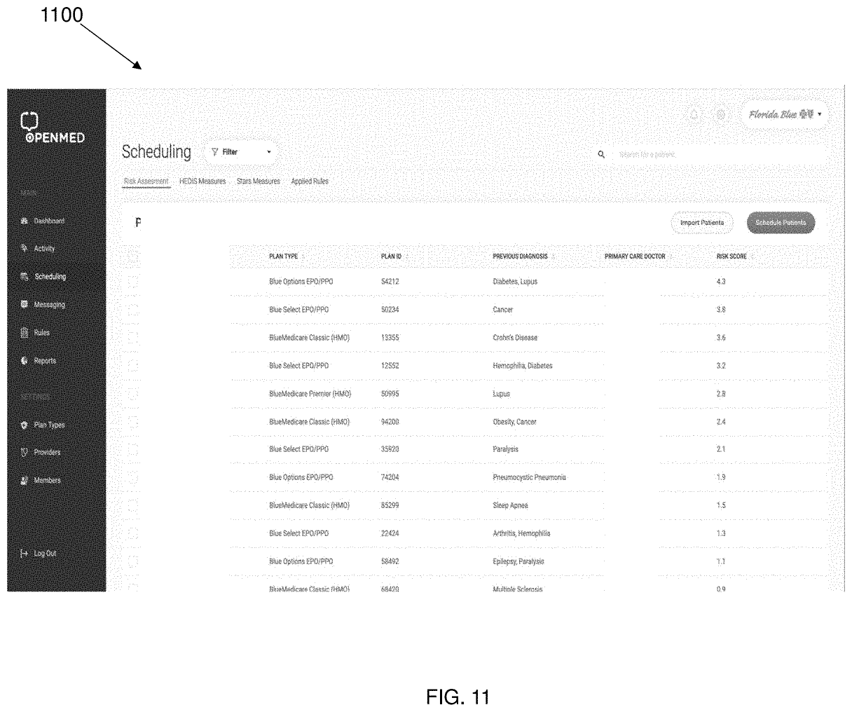

FIGS. 10 and 11 show other interfaces 1000 and 1100, respectively, which in these cases include medical information of the patient and measures taken with respect to the medical information, e.g., last time seen, medications, tests performed, results of the tests, etc. The interface 1000 of FIG. 10 can also include a risk score associated with the patient. This risk score can be a numerical value that provides a risk assessment of a negative event occurring to the patient, which may be used to determine the frequency of notifications and requested appointments. For example, a high score may represent a high risk of a medical event, which would prompt the system to provide additional or more frequent notifications to the patient. The risk assessment can change depending on the changed circumstances of the patient, e.g., taking medications on a regular basis, had a negative test result, etc.

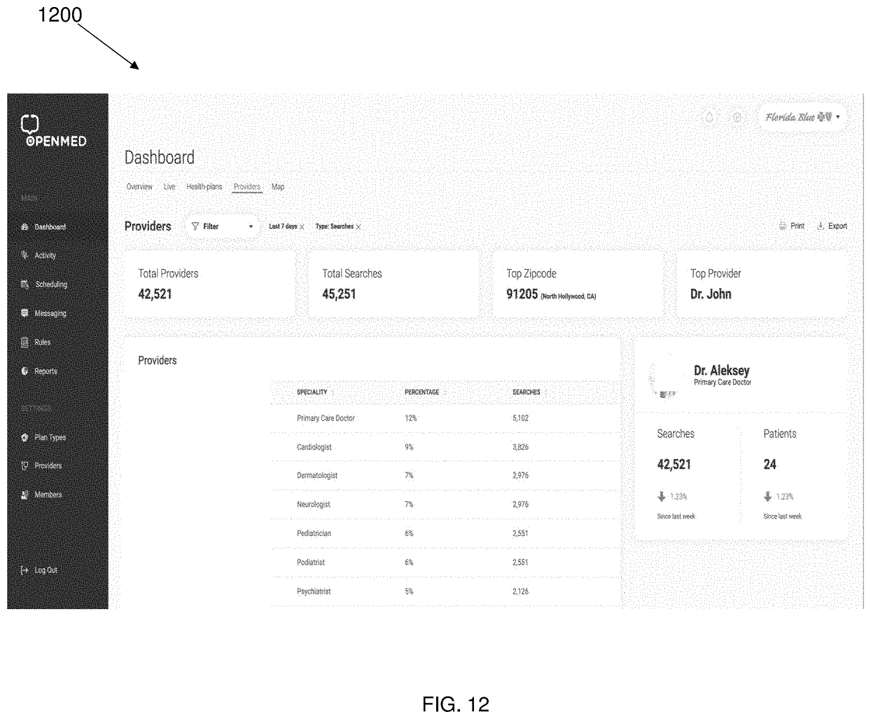

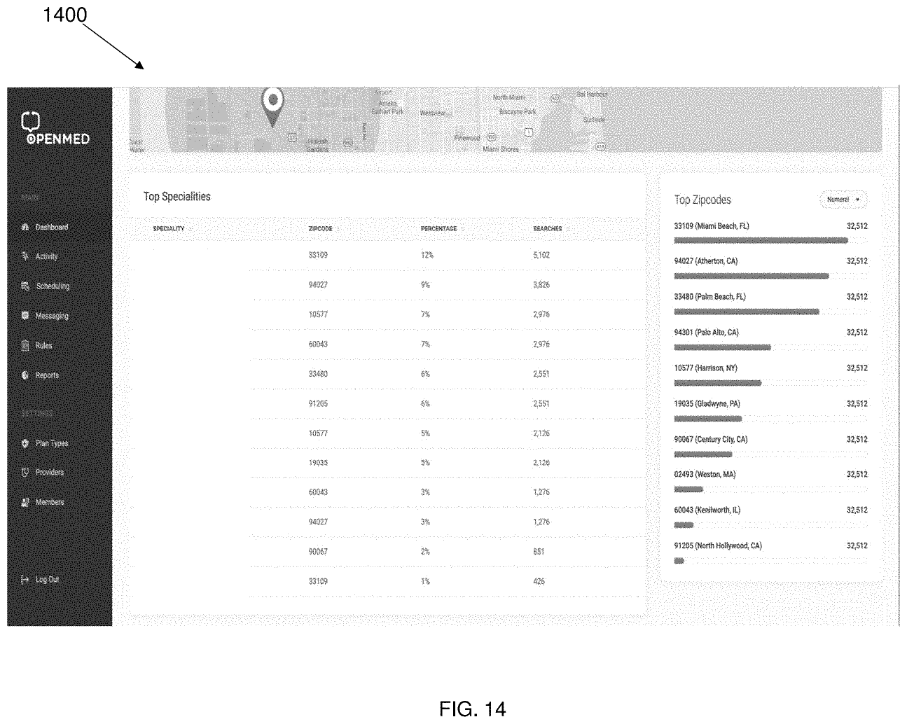

FIGS. 12 and 14 show interfaces 1200 and 1400, respectively, listing providers and their specialty. The interface can be filtered by many different criteria such as zip code, specialty, patient subset, etc.

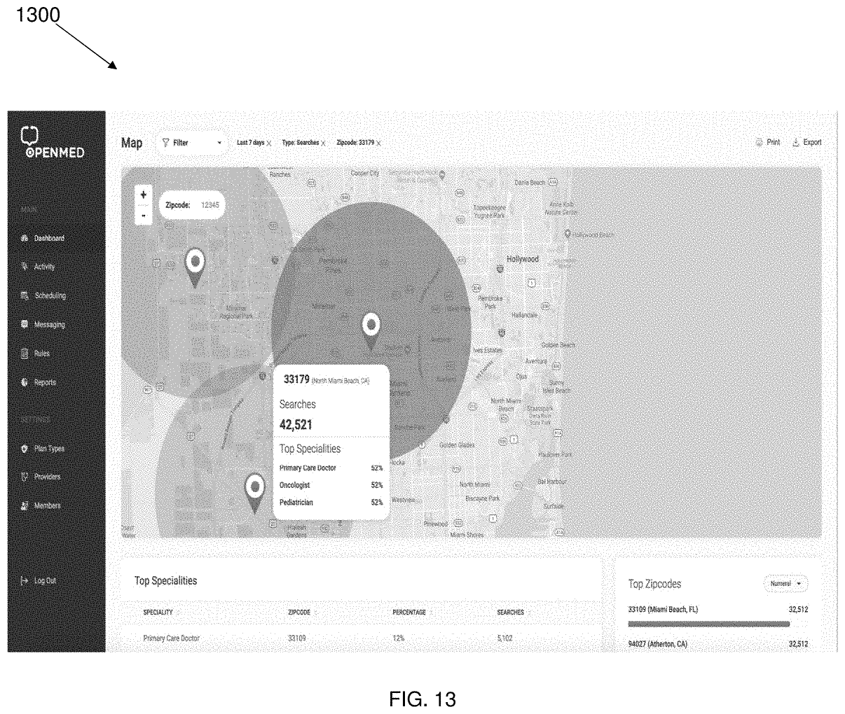

FIG. 13 shows an interface 1300 which provides map information. The map information can be used to graphically show and/or determine location of physicians for certain patients and within certain locations. The maps can be dynamically adjusted, as with all interfaces, based on filtering criteria. To this end, the information in this and other interfaces can be filtered as described herein to provide a subset of patient and/or provider information.

With regard to the interfaces described above, it should thus be understood that the interfaces can be used to build a filter on a population based on a condition which the provider/third party wishes to schedule an appointment. The interfaces can be dynamic, i.e., can be changed depending on new patient information, new provider information, or a host of other criteria. The interfaces each have a filter, which can be used to select automatically a subset of patients. The subset of patients can then be notified to schedule an appointment utilizing the system and processes described herein. Other interfaces could be used to show and schedule available times for appointments.

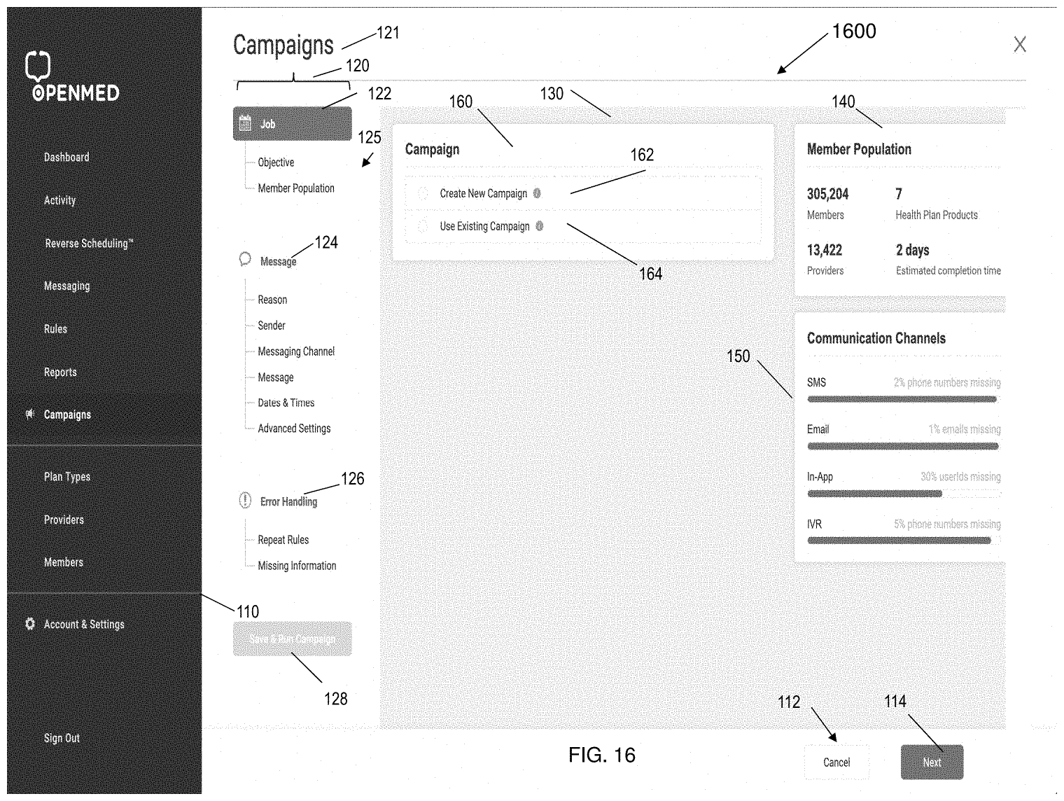

In each of FIGS. 16-25, the interface includes a main navigation sidebar menu 110, a current display sidebar 120, an information and operations window 130, a member population status box 140, a communication channels status box 150 and a bottom navigation bar 112, including a “Next” button 114.

In FIGS. 16-25, the main navigation sidebar menu 110 provides a listing of available services and processes for the application (in the example shown in FIGS. 16-25, the OpenMed™ Web App), including, for example, a dashboard, activity, Reverse Scheduling™, messaging, rules, reports, campaigns, plan types, providers, members, and account & settings. In FIGS. 16-25, the user has selected “Campaigns” as the application which is currently being run. The selected application is shown above the current display sidebar 120 as the current selected application indicator 121 so that the currently selected application is readily apparent to the user. Selections from the main navigation sidebar menu 110 for other applications, such as messaging, members, scheduling and dashboard, are shown, for example, in FIGS. 6 and 8-13.

The current display sidebar 120 in FIGS. 16-25 identifies what the information currently displayed in the information and operations window 130 pertains to, for example, job, message, error handling and “save & run campaign.” Under the job heading 122, subheadings regarding the objective of the job and member population pertaining to the job are shown, and correspond to information provided in the information and operations window 130. Under the message heading 124, the subheadings regarding reason for the message, the sender of the message, the messaging channels, the message itself, dates and times and advanced settings are provided. Under the error handling heading 126, the subheadings of repeat rules and missing information are provided.

The current display sidebar 120 in FIGS. 16-25 includes boxes 122, 124, 126 and 128 to indicate which operation is currently being carried out. When the job heading 122 is highlighted, it indicates that the current operation is the job operation, specifically shown with subheadings for objectives and member population for the particular selected job. When the message box 124 is highlighted, this indicates that the current operation pertains to creating of a message, including subheadings for reasons that can be added to the message, who will appear as the sender of the message, which message channels will be used, the actual message itself, selection of the dates and times, and advanced settings.

When the error handling box 126 is highlighted, this indicates that the current operation pertains to setting parameters for handling errors such as establishing repeat rules for resending the message if necessary, and what information is determined to be missing. When the box 128 is highlighted, this indicates that the current operation pertains to saving and running the campaign. The relationship of the subheadings under the headings 122, 124 and 126 to the information and operations displayed in the information and operations window 130 will be discussed below with regard to FIGS. 16-25.

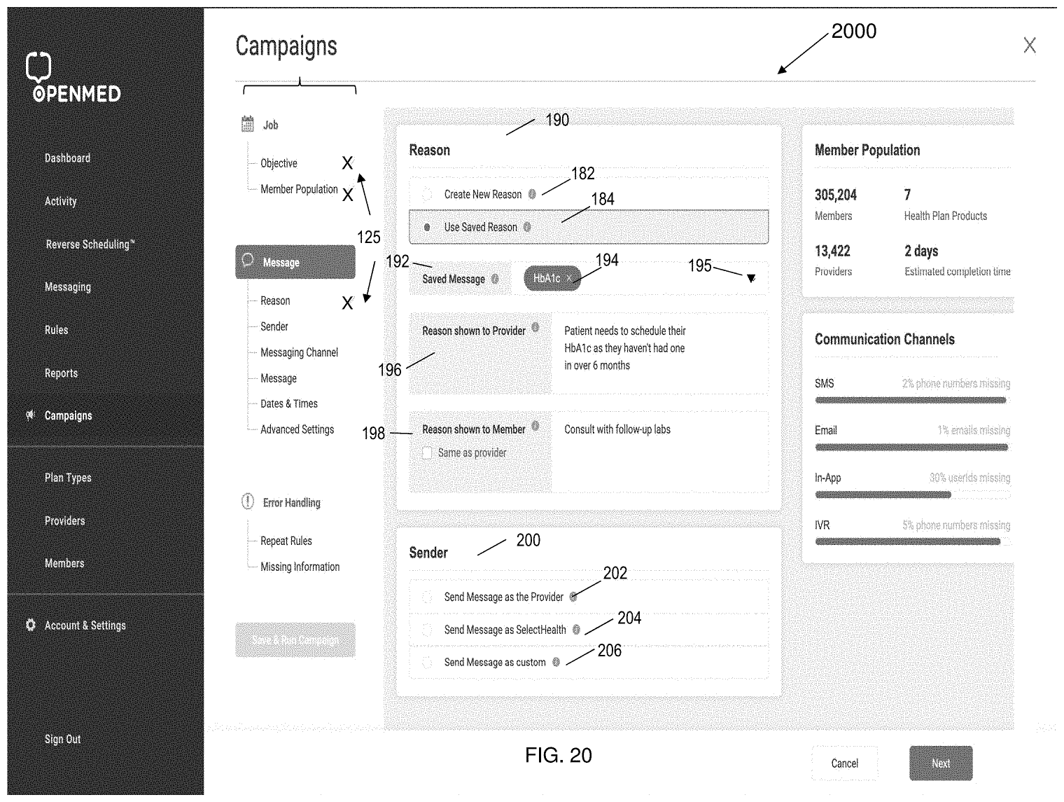

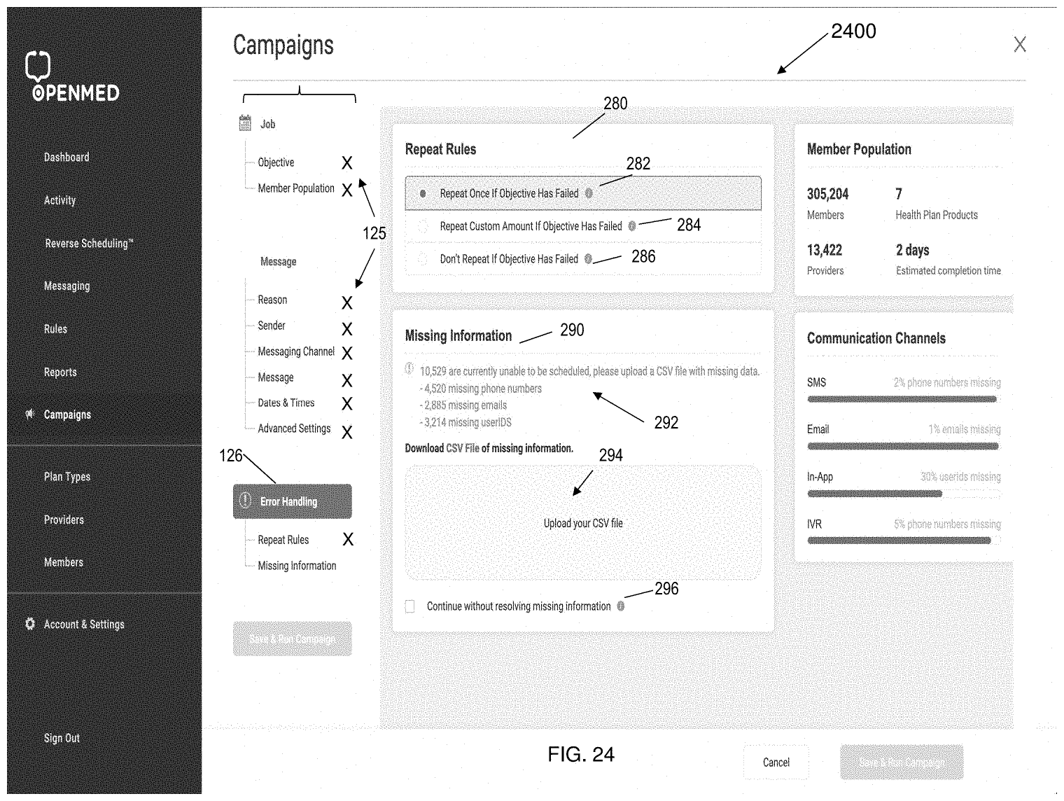

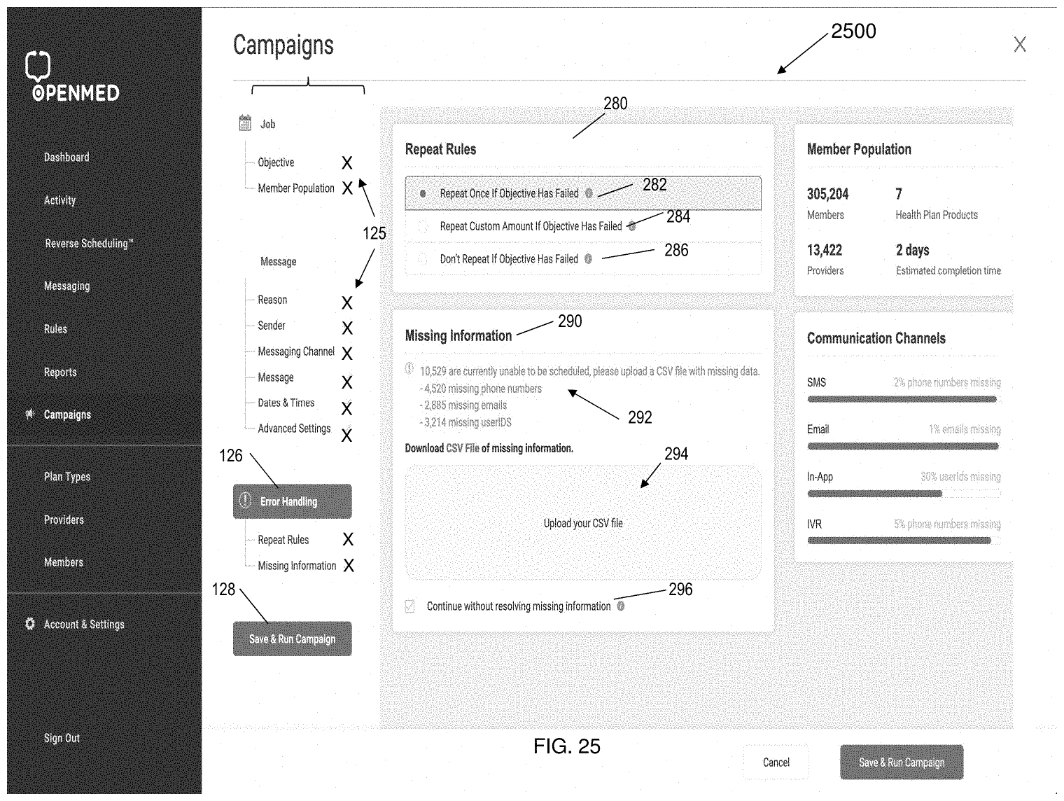

As can be appreciated from the changes shown in the highlighting of the respective boxes 122, 124, 126 and 128 in the current display sidebar 120 in FIGS. 16-25, the current display sidebar 120 allows the user to see the progression form the selection of objectives and member population at the beginning of the campaign (indicated by highlighting the box 122 in FIGS. 16-19), to creating of the message or messages for the campaign (indicated by highlighting box 124 in FIGS. 20-23), to error handling (indicated by highlighting box 126 in FIGS. 24 and 25), to saving and running the campaign (as indicated by highlighting box 128, as shown in FIG. 25).

In accordance w aspects of the disclosure, a progressive checklist area 125 is provided in the current display sidebar 120 to indicate to the user what operations have been completed. This gives the user an immediate indication of the progress in creating the campaign, i.e., what has been completed and what remains to be completed.

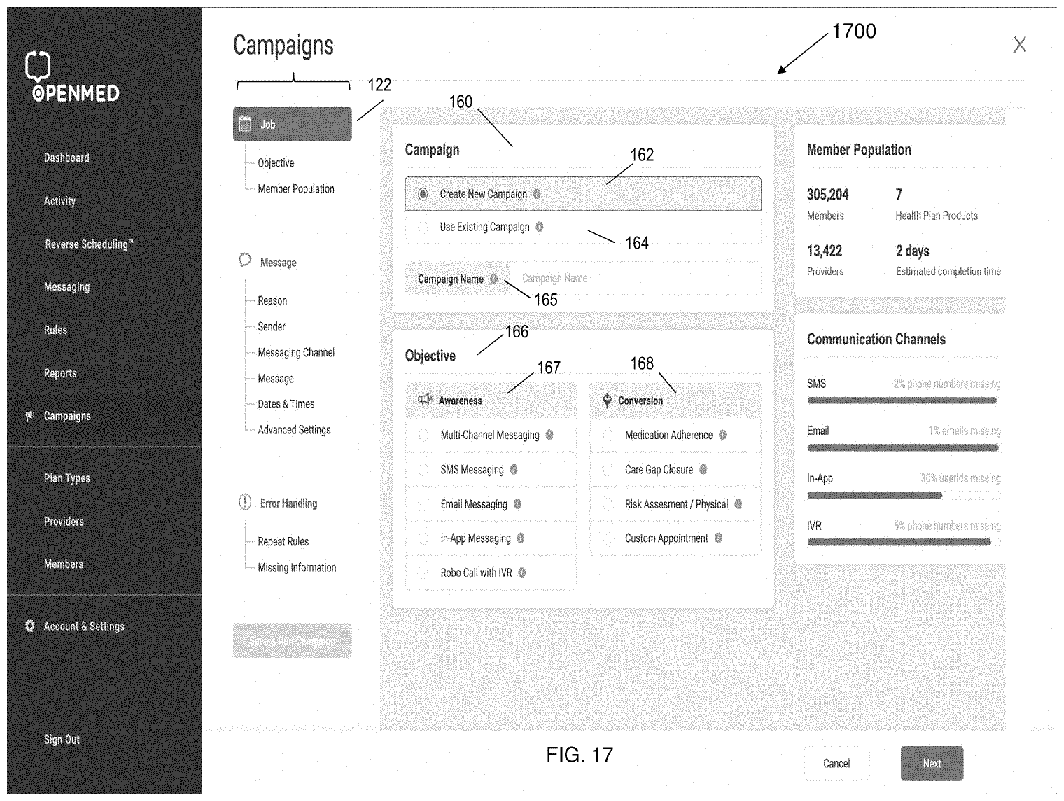

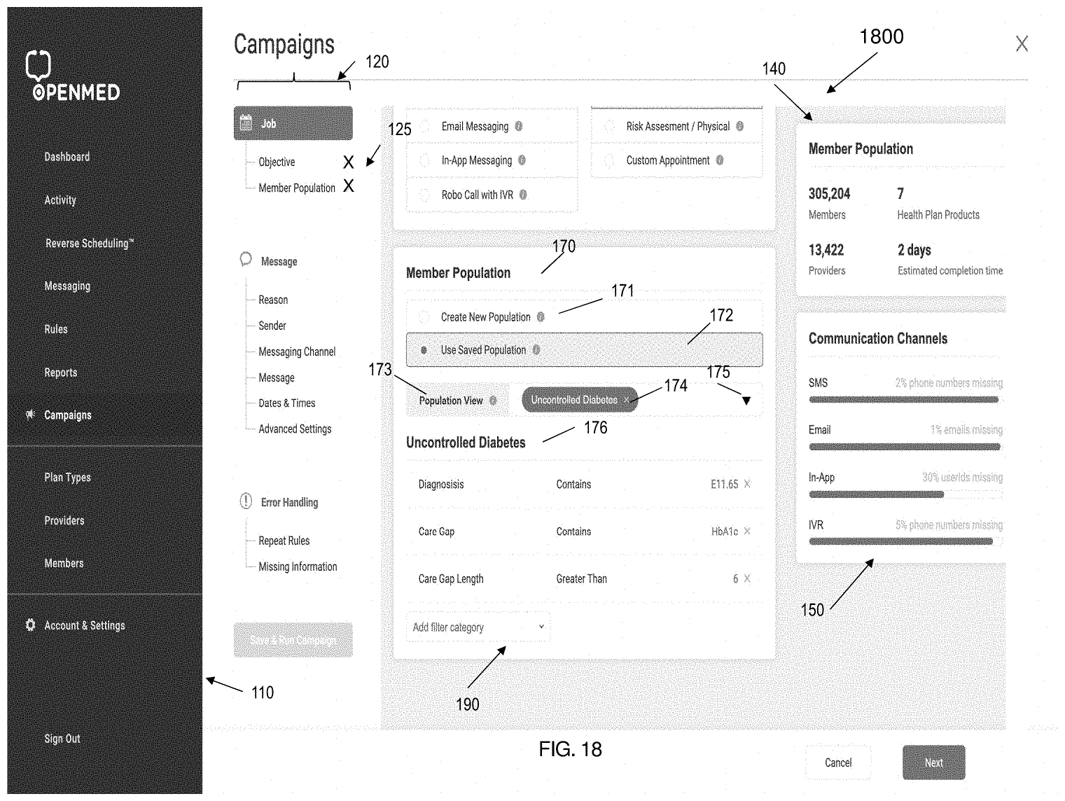

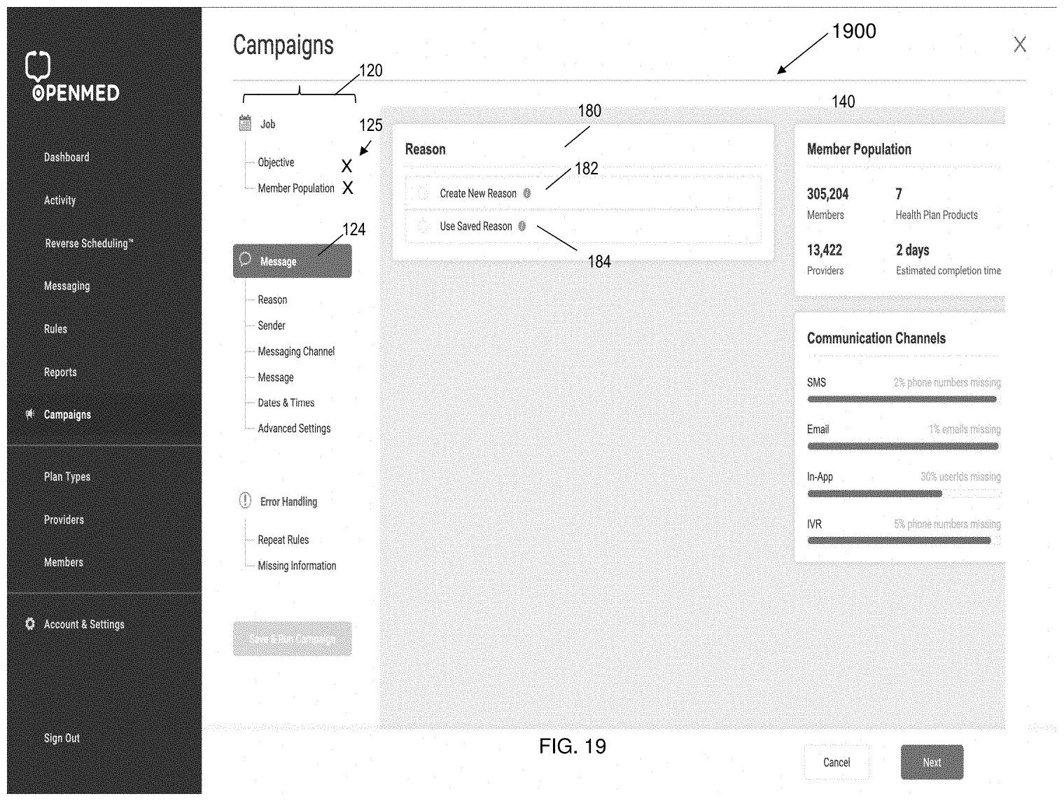

Specifically, this can be appreciated by referring to the progressive checklist area 125 for each of FIGS. 16-25. In FIGS. 16 and 17, when the user is just beginning the creation of the campaign, there are no check marks (indicated in the drawings with “x”) since the operations are just beginning. However, in FIG. 18, boxes have checked for both the objectives and member population because, in the interface shown in FIG. 18, these operations have now been completed, as will be discussed below. After the progress reached by FIG. 18, each of the FIGS. 19-35 shows the progress of the operations through the creation of the campaign by continuing to check off the progressive checklist area 125 as the user continues in creating the campaign.

The information and operations window 130 displays current information, corresponding to the status of the current display sidebar 120, and provides controls, such as checkboxes and drop-down menus, for determining operations. For example, as will be discussed with regard to FIGS. 16-25, the controls provided in the information and operations window 130 allow the user to implement a campaign for a service provider to contact selected members of the member population in accordance with the reverse scheduling aspects of the present disclosure, as will be discussed below with regard to FIGS. 16-25.

The member population status box 140 shown in each of FIGS. 16-25 provides up-to-date information for the user to determine the current number of members, the current number of health plan products, the current number of providers and, in the case of running a campaign in accordance with the selected service of “Campaigns” by the user in the main navigation sidebar menu 110, the estimated completion time for the campaign being planned and implemented. In the case of the particular example discussed below with regard to FIGS. 16-25 for providing a messaging campaign to selected members in the member population for the selected condition of uncontrolled diabetes, the estimated completion time is shown in the member population status box 140 as two (2) days. This estimated completion time can, of course, vary depending on the particular condition which the user selects for the messaging campaign, as well as the total number of members in the member population, the total number of providers, and the total number of health plan products involved. The estimated completion time is also correlated to the particular communication channels selected.

The communication channels status box 150 shows the current state of the various types of communication channels which are available to the user. This current state of the various communication channels is useful for the user to determine the condition of the different types of communication channels available for carrying out the campaign. For example, in the example shown in FIGS. 16-25, it can be seen that in the SMS communication channel, 2% of the phone numbers are missing, in the email communication channel, 1% of the email addresses are missing, for the In-App communication channel, 30% of user IDs are missing, and for the IVR communication channel, 5% of the phone numbers are missing. As such, the communication channels status box 150 allows the user to quickly determine which communication channels will have the best likelihood of reaching the largest number of the selected members for the particular campaign being planned and run.

Turning to the individual interfaces shown in FIGS. 16-25, each of these interfaces shows an exemplary interface in an operation for:

(i) determining whether a new campaign or an existing campaign will be used;

(ii) determining the objectives of the campaign;

(iii) determining the member population to direct the campaign to a particular condition;

(iv) determining reasons for the campaign, and allowing the user to provide the reasons to a provider and, if appropriate, to the members themselves;

(v) determining who the messages will be shown as being sent from;

(vi) determining the desired messaging channel;

(vii) creating a message, or using a saved message;

(viii) selecting dates and times for the message;

(ix) determining repeat rules if the message fails; and

(x) saving and running the campaign.

FIG. 16 shows an interface that provides the user the opportunity to select box 162 in a campaign box 160 to create a new campaign or select box 164 to use an existing campaign. In the example, the user selects “Create New Campaign” in box 162, and clicks the “Next” button 114 in the bottom navigation bar 112 to proceed to the interface shown in FIG. 17.

FIG. 17 provides the user with a display in the information and operations window 130 to create the new campaign. In particular, in FIG. 17, the user is provided with the opportunity to create a campaign name, and to make a selection of objectives. Under the subheading “Conversion”, the user can select an objective, in this example, of “Medication Adherence,” “Care Gap Closure,” “Risk Assessment/Physical,” or “Custom Appointment,” noting that these objectives are solely for purposes of example, and not intended to limit the present disclosure. Under the subheading of “Awareness,” the user is also provided with a selection of possible messaging types to select for obtaining the objective. In the particular example of FIG. 17, the user selects “Care Gap Closure,” and then clicks the “Next” button 114 in the bottom navigation bar 112, which brings up the interface shown in FIG. 18. Alternatively, the user can scroll from the interface shown in FIG. 17 to the interface shown in FIG. 18.

FIG. 18 is an interface which can be used to confirm filtering of patient information regarding the member population to tailor the campaign to a particular condition. In this regard, in FIG. 18, the user has selected “Campaigns” in the main navigation sidebar menu 110 to obtain the particular interface shown in FIG. 18. For example, when “Campaigns” is selected by the user, the current display sidebar 120 shows that the display in the information and operations window 130 corresponds to “Job,” and includes the objective and the member population (noting that, for clarity, the information and operations window 130 in FIG. 18 shows the entire member population box 170, but only a lower portion of the objective box 160, which is shown in more detail in FIG. 17.

In FIG. 18, in the information and operations window 130, the user has selected “Use Saved Population” box 172 in the Member Population box 170 of the information and operations window 130, which has, in turn, displayed the saved population for “Uncontrolled Diabetes” in a selected condition display box 176. Alternatively, the user could select the “Create New Population” box 171 which would display a blank form for the user to populate to create a new population.

The information and operations window 130 further shows that the selected saved member population is for members within the overall member population who have a diagnosis containing E11.65 (which is a diagnosis code for patients with type II diabetes mellitus with hyperglycemia), and a care gap containing HbA1c (i.e., which is an indicator of the average level of blood sugar shown in a hemoglobin A1c test) and a care gap length of greater than six months.

The interface shown in FIG. 18 also includes a population view indicator 173, as a heading for the selected condition display box 176, and a current population view description box 174, which includes a drop-down menu button 175 to select a population from a list of saved populations. The example shown in FIG. 18, the particular saved population selected by the user is for “Uncontrolled Diabetes,” as shown in the selected condition display box 176.

It should be understood by those of skill in the art that other medical conditions, diagnoses codes, care gaps, etc. can be utilized with the interface shown in FIG. 18, and that the particular example shown is not intended to be limiting in any way with respect to the functionality and features of the present disclosure.

With regard to the saved population utilized in the example shown in FIG. 18, this particular saved population can be created by selecting “Reverse Scheduling™” in the main navigation sidebar menu 110, importing patients from the overall member population, and filtering the members by selecting the particular condition (in this example, uncontrolled diabetes with a diagnosis containing E11.65), and by also filtering for the care gap (containing HbA1c) and a desired care gap length (in the example, greater than six months). It is to be understood, of course, that any desired condition, and details of that condition, can be used for the filtering to obtain the selected member population for the campaign in accordance with aspects of the present disclosure. It is further understood that the details of the care gap and the care gap length can also be adjusted appropriately depending upon the selected condition. Once the user has prepared a member population for the selected condition, this population can be saved so that it will be available to the user when the user selects “Campaigns” in the main navigation sidebar menu 110.

After the “Use Saved Population” box 172 has been checked in the information and operations window 130 in FIG. 18, and after it is verified that this is an appropriate member population for the particular campaign, the user can click the “Next” button 114 in the bottom navigation bar 112 to proceed to the interface shown in FIG. 19. It is to be noted that once the particular member population box 170 is completed by the user (e.g., in this case, by selecting a saved population for uncontrolled diabetes, as shown in FIG. 18), the progress checklist area 125 indicates with checkmarks, or X's, that the operations of determining the objective and the member population, shown under the job heading 120, have been completed. Alternatively, these subheadings could be highlighted as they are completed.

FIG. 19 allows the user to select a reason as to why a message is being sent in the current campaign. This is particularly useful for a provider to understand why the message is important. In other words, the reason is useful for the provider to understand why the campaign is being implemented. Optionally, the reason, or a different reason, can also be provided to the members receiving the message as well, if this is determined by the user creating the campaign to be appropriate.

For the purpose of allowing the user to select a reason for the message, the interface shown in FIG. 19 provides a reason box 180. The reason box 180 includes subheading box 182, if the user wishes to create a new reason for sending the message, and subheading box 184, if the user prefers to use a saved reason for sending the message. It is noted that in changing the interface display from FIG. 18 to FIG. 19, the application converts from operating with regard to determine objectives and member population, under the job heading 122, indicated in the current display sidebar 120, to the message heading 124 (which is now highlighted). This provides a convenient indication to the user that the current interface being displayed in FIG. 19 pertains to the creation of the message used in the campaign, rather than establishing objectives or the member population for the campaign.

As noted above, the reason itself pertains to either a reason to be shown to the provider and, optionally, to members receiving the campaign message, as to why the message is being sent, as will be discussed further below with regard to FIG. 20. For purposes of the current example, the user selects subheading box 184 to use a saved reason, and then clicks the “Next” button 114 in the bottom navigation bar 112 to proceed to the next interface shown in FIG. 20.

The interface shown in FIG. 20 provides a detailed reason box 190, allowing the user to see a saved reason for possible use in the campaign, in accordance with the user having selected the “Use Saved Reason” box 184 in the interface shown in FIG. 19. The interface shown in FIG. 20 also provides a sender box 200 allowing the user to select whether the campaign message will be sent to the members with an indication that it is being sent as a message from the provider (subheading box 202), or from a third-party other than a provider (subheading box 204, in this example, indicating a network provider SelectHealth™), or from a customized sender (subheading box 206) selected by the user.

The detailed reason box 190 in the example shown in FIG. 20 highlights the “Use Saved Reason” box 184, indicating that this was the selection made by the user in the interface shown in FIG. 19. Based upon the selection of using a saved reason, a “Saved Message” box 192 indicates a message which has previously been saved for the selected condition box 194 of “HbA1c” which, as previously noted, corresponds to an indication of uncontrolled diabetes, as discussed with regard to FIG. 18.

In the example shown in FIG. 20, the saved messages 192 for the condition HbA1c shown in the condition box 194 is shown in the “Reason shown to Provider” box 196 and “Reason shown to Member” box 198. Specifically, for this particular condition, the reason which will be provided to the provider in the box 196 is “Patient needs to schedule their HbA1c as they haven't had one in over six months.” On the other hand, the reason which will be shown to the member is “Consult with follow-up labs,” as shown in the box 198. It is noted that the box 198 provides a checkbox “Same as Provider” if the user determines that it is appropriate for the member to see the same message which is given to the provider. It is also noted that leaving the “Reason shown to “member” box 198 empty will mean that no reason will be provided to the member in the campaign message. Similarly, the user can opt not to provide a reason to the provider by leaving the box 196 empty.

A drop-down menu box 195 is provided in the detailed reason box 190 to allow the user to view a list of other conditions which have saved messages, for example, from previous campaigns. By clicking on the drop-down menu box 195 and selecting a different condition, the selected different condition will be shown in the selected condition box 194, and the displayed messages will, correspondingly, be changed for the boxes 196 and 198.

It is noted that if the user had selected the “Create New Reason” box 182, the detailed reason box 190 would provide empty versions of the “Reason shown to Provider” box 196 and the “Reason shown to Member” box 198, to allow the user to create new reasons. Also, the sender box 200 would be provided to allow the user to select who the message will appear to be from. However, providing the “Saved Message” box 192 and the selected condition box 194, with the drop-down menu box 195, would be optional in this case. If the “Saved Message” box 192 is provided in the detailed reason box 190 when the “Create New Reason” box 182 is selected, it would not be highlighted if the box 182 is selected and highlighted.

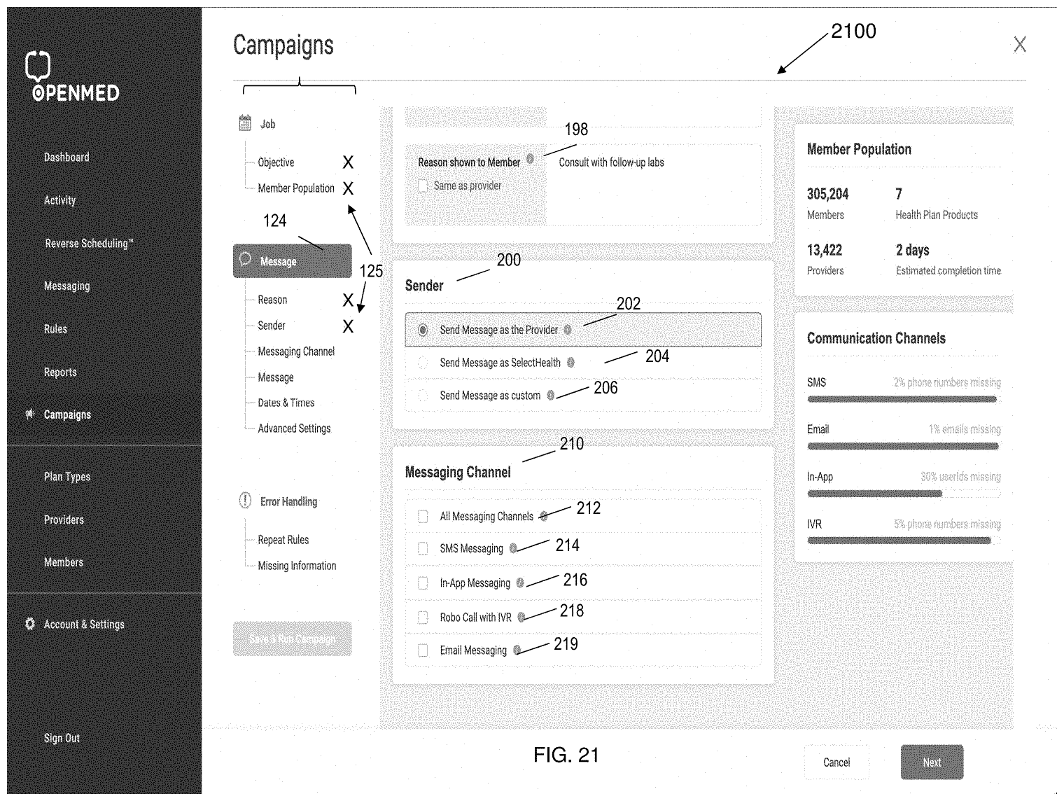

Once the user has determined that the reasons shown in the boxes 196 and 198 are satisfactory, and has made a choice from the three options provided by the send message boxes 202, 204 and 206, clicking the “Next” button 114 in the bottom navigation bar 112 will bring up the interface shown in FIG. 21. In the example provided here, the user selects the “Send Message as the Provider” box 202 on the interface shown in FIG. 20. It is noted that, in FIG. 20, the progress checklist area 125 shows that, in addition to the checked boxes for objective and member population under the job heading 122 of the current display sidebar 120, a checkmark or “X” is now shown next to the “reason” subheading under the message heading 124.

FIG. 21 shows the interface for allowing the user to select one or more messaging channels for the campaign message. To this end, a messaging channel box 210 is provided, which, in this example, gives the option of “All Messaging Channels” box 212, “SMS Messaging” box 214, “In-App Messaging” box 216, “Robo Call with IVR” box 218 and “Email Messaging” box 219. The user can select one or more of the boxes 214, 216, 218 and 219, or simply check the box 212 to include all of the possibilities listed in the boxes 214, 216, 218 and 219. It is noted that FIG. 21 shows the “Send Message As the Provider” box 202 highlighted, indicating the selection of this option by the user in the interface shown in FIG. 21. It is also noted that FIG. 21 shows the lower portion of the detailed reason box 190 shown in FIG. 20. In this regard, it is noted that FIG. 21 can be reached, in accordance with embodiments of the disclosure, by scrolling down from the interface shown in FIG. 20.

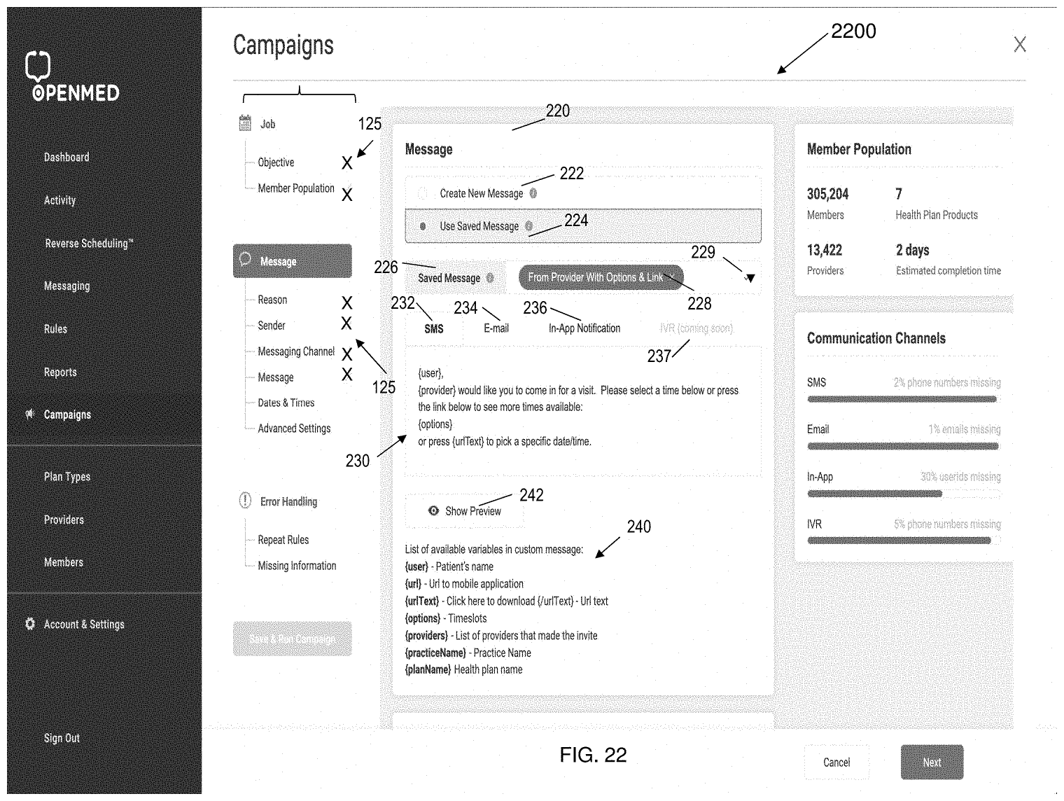

In the current example, the user selects the “All Messaging Channels” box 212. Once the user has selected the desired one or more messaging channels from the messaging channel box 210, clicking on the “Next” button 114 in the bottom navigation bar 212 brings up the interface shown in FIG. 22. It is noted that in FIG. 21 a checked box is added in the progress checklist area 125 next to the subheading of “Sender” under the message heading 124.

FIG. 22 provides the user with the message creation box 220 which allows the user to prepare the actual message which will be used in the campaign. For this purpose, the message creation box 220 includes box 222 to allow creation of a new message and box 224 to use a saved message. In the example shown in FIG. 22, the user selects box 224 to use a saved message, and this highlights the box 226 indicating the selection of a saved message. With regard to the saved message, the box 228 provides a list of saved messages, with a drop down menu box 229 to select from a stored list of such saved messages. In the example shown in FIG. 22, the user has selected a saved message for “From Provider With Options & Link” from the list of available options that appear when the scroll down box 229 is selected. This saved message is shown in the message box 230, along with a list of available variables 240 for customizing the message shown in the message box 230. This allows the user the opportunity to customize the actual message which will be sent.

FIG. 22 also shows tabs 232, 234 and 236, where the tab 232 is for SMS messages, the tab 234 is for email messages, and the tab 236 is for in-app notifications. In the example shown, the message being composed in the message box 230 at the time is for SMS messaging, indicated by the highlighting of the SMS tab 232. In accordance with aspects of the present disclosure, the user can click on the email tab 234 to compose an email message, and click on the in-app notification tab 236 to create an in-notification message. In this way, different messages can be provided for the different communication channels, if desired. Alternatively, the user can utilize the same message for each of the different selected communication channels.

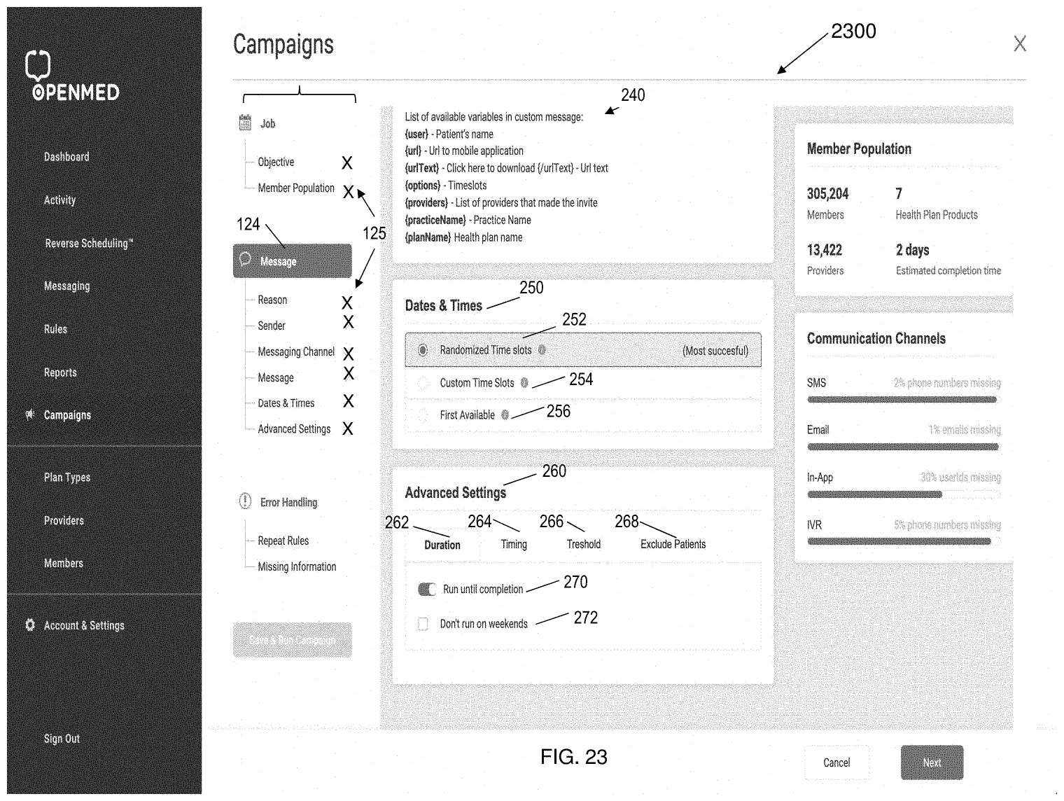

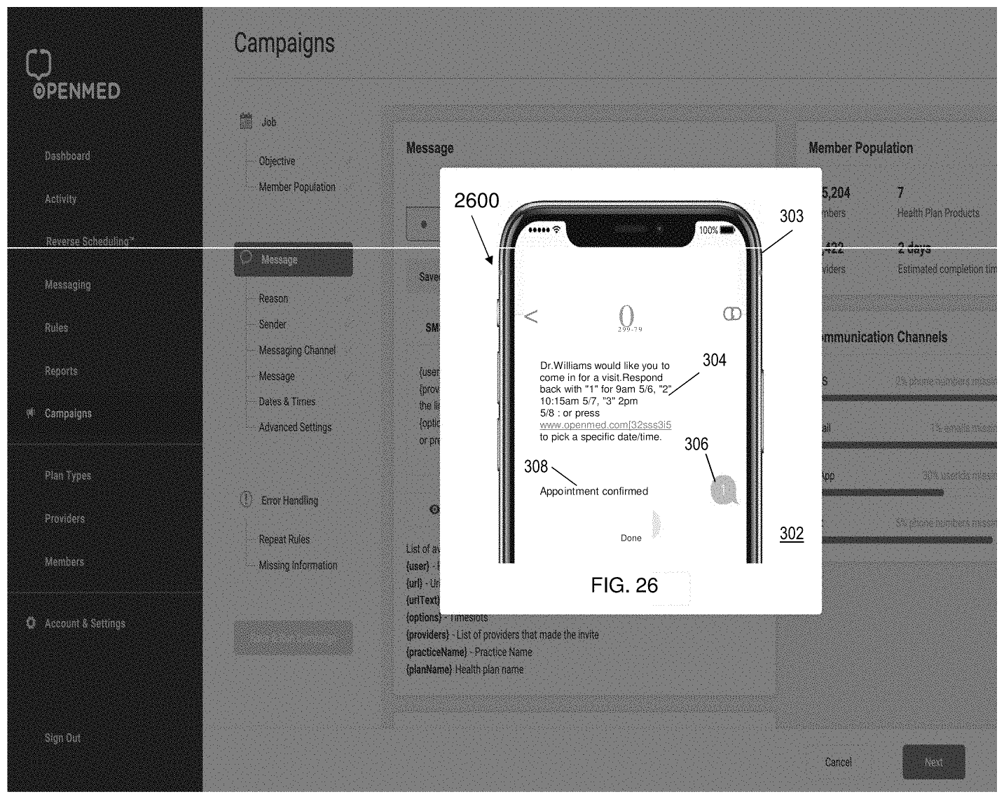

In accordance with another aspect of the present disclosure, a show preview box 242 is provided. This show preview box 242 provides the user with the option of seeing a preview of what the message will look like on a user device, as shown in FIG. 26., which will be discussed hereinafter. After the user has completed preparation of the message in the message box 230, clicking on the “Next” box 114 in the bottom navigation bar 112 brings up the interface shown in FIG. 23. In alternatives of the present disclosure, the user can scroll down to the interface shown in FIG. 23. It is noted that in FIG. 22, checkmarks, or “X's” are added in the progress checklist area 125 next to the subheadings of “Messaging Channel” and “Message” under the message heading 124.

FIG. 23 allows the user to provide timeslots for appointments in the message for members receiving the message to choose from. For this purpose, a “Dates & Times” box 250 is provided. This box 250, in the example shown, provides the user with the options of indicating randomized timeslots (box 252), custom timeslots (box 254) or the first available appointment (box 256). As shown in FIG. 23, the box 252 indicates to the user that randomized timeslots are the most successful in securing a response.

In addition, FIG. 23 provides an advanced settings box 260 providing further setting capabilities for the message to be used in the campaign before it is sent. In the example shown, four selection tabs are provided, specifically, tab 262 (for selecting duration of the campaign), tab 264 (for selecting the timing of the campaign), tab 266 (for selecting a threshold at which the campaign will end) and tab 268 (for providing the option of excluding certain patients from the campaign). It is to be understood, of course, that these are just examples of advanced settings, and other settings could be used without departing from the scope of the present disclosure.

In the example shown in FIG. 23, the tab 262 for setting duration of the campaign has been selected, as indicated by the tab 262 being highlighted. Based upon the selection of this tab 262, box 270 is provided to allow the user to select running the campaign until completion, and box 272 is provided to allow the user the option of not running the campaign on weekends. Of course, the number of tabs and the settings which they permit shown in the example of FIG. 23 is solely for purposes of example, and not intended to be limiting to the present disclosure. In the example shown in FIG. 23, it is noted that the user has selected the randomized timeslots box 252 and the option for running until completion 274 under the selected duration tab 262.

Once the user has made a selection with regard to dates and times and advanced settings, clicking the “Next” button 114 in the bottom navigation bar 112 brings up the interface shown in FIG. 24. It is noted that with the completion of the options shown in the interface of FIG. 23, all subheadings for the message heading box 124 are completed, and all subheadings in the progress checklist area 125 are checked under the message heading box 124. In accordance with this, the interface shown in FIG. 24 highlights the error handling box 126 to indicate that this stage of the creation of the campaign has now been reached.

In the interface shown in FIG. 24, a repeat rules box 280 and a missing information box 290 are provided. The repeat rules box 280 provides the user with three options for repeating sending of the message if the objective has failed (generally in that some of the members selected for the campaign have not responded). Specifically, the repeat rules box 280 provides subheading boxes 282, 284 and 286 in the example shown. Box 282 sets the campaign to repeat once if the objective has failed. Box 284 allows the user to set a custom amount for repeated sending of the message if the objective has failed. Box 286 allows the user to decide not to repeat sending a message if the objective has failed. As such, the repeat rules box 280 allows a user a wide variety of possibilities for determining how many times to repeat sending a message to non-responding selected members for the campaign.

The missing information box 290 provides a display 292 for the user with regard to missing information regarding carrying out the campaign. For example, in the display 292 shown in FIG. 24, the user is advised that 10,529 members are currently unavailable to be scheduled, together with a recommendation to upload a CSV file missing data. The display 292 also indicates that there are 4,520 missing phone numbers, 2,885 missing emails and 3,214 missing user IDs with regard to implementing the campaign for the selected member population (see FIG. 18). Box 294 provides the user with the opportunity to upload the CSV file for purposes of providing missing information. Alternatively, box 296 allows the user to select the option of continuing without resolving the missing information.

Once the user selects one of the boxes 282, 284 and 286 in the repeat rules box 280, progress checklist area 125 shows a check mark, or “X” for the repeat rules subheading under the error handling heading 126. Once the user provides the missing information by uploading the CSV file in box 294 or checkmarks the box 296 to continue without resolving information, the interface shown in FIG. 24 changes to the interface shown in FIG. 25. Specifically, as can be seen from FIG. 25, the user is now provided with box 302 “Save & Run Campaign,” in the bottom navigation bar 112. Also, the “Save & Run Campaign” subheading box 128 in the current display sidebar 120 is highlighted to indicate to the user that the campaign is now ready to launch. Also, the progress checklist area 125 shows a check mark, or “X”, for the missing information subheading under the error handling heading 126 of the current display sidebar 120.

FIG. 26 is an example of a preview 302 of a composed message which the user can review based upon selecting the “Show Preview” box 242 in FIG. 22. It is also an example of what members in the selected member population for the campaign will actually receive. The preview 302 shows a sample user device 303 and a sample 304 of the composed message. This sample composed message shows timeslots which are based upon the user selection of one of the options provided by the boxes 252, 254 and 256 in the interface shown in FIG. 23. In the particular example of FIG. 26, the sample composed message is “Doctor Williams would like you to come in for a visit. Respond back with “1” for 9 am May 6, “2” 10:15 am May 7, “3” 2 pm May 8: or press www.open med.com/32sss3i5 to pick a specific date/time.” In the preview 302, the user response 306 indicates selection of option “1.” In accordance with the reverse scheduling operations disclosed herein, the application reviews whether this selected option is still available and, if so, provides a sender response 308 indicating “Appointment confirmed.” Of course, if the timeslot in question is no longer available, the sender response 308 would request another selection.

### Computing Infrastructure

The reverse flow appointment scheduling may be implemented as a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium is non-transitory and may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium (non-transitory devices, etc.) includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices as shown in FIG. 15, from a computer readable storage medium or to an external computer or external storage device via a network, for example, the Internet, a local area network, a wide area network and/or a wireless network. Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages.

The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet). In embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general purpose computer, special purpose computer, or other programmable data processing apparatus (all of which are representative of the computer infrastructure shown in FIG. 15), such that the instructions, which execute via the processor of the computer or other programmable data processing apparatus, create means for implementing the functions/acts specified in the flow diagrams and/or block(s) thereof. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

Referring now to FIG. 15, a schematic of an example of a computer infrastructure is shown. Computer infrastructure 10 is only one example of a suitable computer infrastructure and is not intended to suggest any limitation as to the scope of use or functionality of embodiments of the invention described herein. Regardless, computer infrastructure 10 is capable of being implemented and/or performing any of the functionality set forth hereinabove.

In computer infrastructure 10 there is a computer system 12, which is operational with numerous other general purpose or special purpose computing system environments or configurations. Examples of well-known computing systems, environments, and/or configurations that may be suitable for use with computer system 12 include, but are not limited to, personal computer systems, server computer systems, thin clients, thick clients, hand-held or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system 12 may be described in the general context of computer system executable instructions, such as program modules, being executed by a computer system. Generally, program modules may include routines, programs, objects, components, logic, data structures, and so on that perform particular tasks or implement particular abstract data types. Computer system 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 15, computer system 12 in computer infrastructure 10 is shown in the form of a general-purpose computing device. The components of computer system 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computer system 12 typically includes a variety of computer system readable media. Such media may be any available media that is accessible by computer system 12, and it includes both volatile and non-volatile media, removable and non-removable media.

System memory 28 can include computer system readable media in the form of volatile memory, such as random access memory (RAM) 30 and/or cache memory 32. Computer system 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Program modules 42 generally carry out the functions and/or methodologies of embodiments of the invention as described herein.

Computer system 12 may also communicate with one or more external devices 14 such as a keyboard, a pointing device, a display 24, etc.; one or more devices that enable a user to interact with computer system 12; and/or any devices (e.g., network card, modem, etc.) that enable computer system 12 to communicate with one or more other computing devices. Such communication can occur via Input/Output (I/O) interfaces 22. Still yet, computer system 12 can communicate with one or more networks such as a local area network (LAN), a general wide area network (WAN), and/or a public network (e.g., the Internet) via network adapter 20. As depicted, network adapter 20 communicates with the other components of computer system 12 via bus 18. It should be understood that although not shown, other hardware and/or software components could be used in conjunction with computer system 12. Examples, include, but are not limited to: microcode, device drivers, redundant processing units, external disk drive arrays, RAID systems, tape drives, and data archival storage systems, etc.

In embodiments, a service provider could offer to perform the processes described herein. In this case, the service provider can create, maintain, deploy, support, etc., the computer infrastructure that performs the process steps of the invention for one or more customers. These customers may be, for example, any business that uses technology. In return, the service provider can receive payment from the customer(s) under a subscription and/or fee agreement and/or the service provider can receive payment from the sale of advertising content to one or more third parties.

In still additional embodiments, the invention provides a computer-implemented method, via a network. In this case, a computer infrastructure, such as computer system 12, can be provided and one or more systems for performing the processes of the invention can be obtained (e.g., created, purchased, used, modified, etc.) and deployed to the computer infrastructure. To this extent, the deployment of a system can comprise one or more of: (1) installing program code on a computing device, such as computer system 12, from a computer-readable medium; (2) adding one or more computing devices to the computer infrastructure; and (3) incorporating and/or modifying one or more existing systems of the computer infrastructure to enable the computer infrastructure to perform the processes of the invention.

The systems and processes described herein can also be implemented in a cloud computing environment. Also, embodiments of the systems and processes described herein are capable of being implemented in conjunction with any other type of computing environment now known or later developed. It should be recognized that cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include may characteristics including, e.g., on-demand self-service, broad network access, resource pooling, etc., as is known to those of skill in the art such that no further explanation is required herein.

The systems and processes described herein can also be implemented as Software as a Service (SaaS). This capability allows the consumer or other third party to use the provider's (e.g., OpenMed™) applications running on a cloud infrastructure. The applications are accessible from various client devices through a thin client interface such as a web browser (e.g., web-based e-mail). The consumer need not manage or control the underlying cloud infrastructure including network, servers, operating systems, storage, or even individual application capabilities, with the possible exception of limited user-specific application configuration settings.

The systems and processes described herein can also be Platform as a Service (PaaS). This capability provided to the consumer or other third party is to deploy onto the cloud infrastructure consumer-created or acquired applications created using programming languages and tools supported by the provider (e.g., OpenMed™). The consumer or other third party does not manage or control the underlying cloud infrastructure including networks, servers, operating systems, or storage, but has control over the deployed applications and possibly application hosting environment configurations.

The systems and processes described herein can also be Infrastructure as a Service (IaaS). This the capability provided to the consumer or other third party is to provision processing, storage, networks, and other fundamental computing resources where the consumer is able to deploy and run arbitrary software, which can include operating systems and applications. The consumer or other third party need not manage or control the underlying cloud infrastructure but has control over operating systems, storage, deployed applications, and possibly limited control of select networking components (e.g., host firewalls).

The descriptions of the various embodiments of the reverse flow appointment scheduling has been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method implemented in a computer infrastructure having computer executable code tangibly embodied on a computer readable storage medium having programming instructions operable to:
gather information, using the computer, regarding a consumer;
analyze, using the computer, the user information to determine if a predetermined time has passed since the consumer has had an appointment with a provider for a predetermined service;
prepare, using the computer, a list of available appointment times which are available for the provider to meet with the consumer to provide the predetermined service;
prepare, using the computer, a notification to the consumer with the list of available appointment times; and
send, using the computer, the notification to the consumer to request that the consumer schedules an appointment by selecting one of the appointment times from the list.

2. The method of claim 1, wherein, at least one of the steps of gathering the provider information, analyzing, preparing the list, preparing the notification and sending the notification is performed by a third party other than the consumer.

3. The method of claim 2, wherein the consumer is a healthcare patient and the third party is at least one of: a health insurer; an HMO; a care giver; and a contractually obligated party to the healthcare patient.

4. The method of claim 3, further comprising the third party preparing, using the computer, the notification and a message to at least one of the provider and the patient regarding reasons for the appointment to be scheduled.

5. The method of claim 1, wherein the provider is a medical services provider and the consumer is a patient of the medical services provider.

6. The method of claim 1, wherein the information is gathered for a plurality of consumers, and wherein the analyzing includes filtering the information of the plurality of consumers to determine which consumers have not had an appointment within the predetermined time for the predetermined service.

7. The method of claim 1, wherein steps of claim 1 are provided by a service provider on a subscription, advertising, and/or fee basis.

8. The method of claim 1, wherein software for performing the steps of claim 1 is provided as a service in a cloud environment.

9. A computer program product for providing reverse flow appointment scheduling for healthcare, the computer program product comprising a computer readable storage medium having program instructions embodied therewith, the program instructions being executable by a hardware computer device to cause the hardware computer device to:
gather information regarding a healthcare patient;
analyze the user information to determine if a predetermined time has passed since the healthcare patient has had an appointment with a healthcare provider for a predetermined service;
prepare a list of available appointment times which are available for the healthcare provider to meet with the healthcare patient to provide the predetermined service;
prepare a notification to the healthcare patient with the list of available appointment times; and
send the notification to the healthcare patient to request that the healthcare patient schedules an appointment by selecting one of the appointment times from the list.

10. The computer program product of claim 9, wherein, at least one of the steps of gathering the provider information, analyzing, preparing the list, preparing the notification and sending the notification is performed by a third party other than the healthcare patient and the healthcare provider.

11. The computer program product of claim 10, wherein the third party is at least one of: a health insurer; an HMO; a care giver; or a contractually obligated party to the patient.

12. The computer program product of claim 11, further comprising the third party preparing the notification and a message to at least one of the healthcare provider and the healthcare patient regarding reasons for the appointment to be scheduled.

13. The computer program product of claim 9, wherein the information is gathered for a plurality of healthcare patients.

14. The computer program product of claim 13, wherein the analyzing includes filtering the information of the plurality of healthcare patients to determine which healthcare patients have not had an appointment within the predetermined time for the predetermined service.

15. The computer program product of claim 14, wherein the filtering is performed to limit sending the notifications to healthcare patients have a predetermined condition.

16. A system comprising:
a processor, a computer readable memory, and a computer readable storage medium;
program instructions to gather information regarding a consumer;
program instructions to analyze the user information to determine if a predetermined time has passed since the consumer has had an appointment with a provider for a predetermined service;
program instructions to prepare a list of available appointment times which are available for the provider to meet with the consumer to provide the predetermined service;
program instructions to prepare a notification to the consumer with the list of available appointment times; and
program instructions to send the notification to the consumer to request that the consumer schedules an appointment by selecting one of the appointment times from the list,
wherein, at least one of the steps of gathering the provider information, analyzing, preparing the list, preparing the notification and sending the notification is performed by a third party other than the consumer, and
wherein the program instructions are stored on the computer readable storage medium for execution by the processor via the computer readable memory.

17. The system of claim 16, wherein the consumer is a healthcare patient and the third party is at least one of: the provider; a health insurer; an HMO; a care giver; and a contractually obligated party to the healthcare patient.

18. The system of claim 17, further comprising program instructions for the third party to prepare the notification and a message to at least one of the provider and the healthcare patient regarding reasons for the appointment to be scheduled.

19. The system of claim 16, wherein the information is gathered for a plurality of consumers.

20. The system of claim 19, wherein the analyzing includes filtering the information of the plurality of consumers to determine which consumers have not had an appointment within the predetermined time for the predetermined service.
